(19) United States
(12) Patent Application Publication
(10) Pub. No.: US 2025/0319182 A1
(43) Pub. Date: Oct. 16, 2025

(54) VIRUS-SPECIFIC T CELLS, METHODS OF THEIR PREPARATION AND USE THEREOF

(71) Applicants: SHEBA IMPACT LTD., Ramat Gan (IL); MINISTRY OF HEALTH, Jerusalem (IL)

(72) Inventors: Gal CAFRI, Nir David (IL); Elad JACOBY, Tel Aviv (IL); Amihai LIEBERMAN, Atlit (IL); Neta ZUCKERMAN, Tel Aviv (IL); Nira VARDA-BLOOM, Hod Hasharon (IL)

(21) Appl. No.: 18/871,611

(22) PCT Filed: Jun. 6, 2023
(86) PCT No.: PCT/IL2023/050580

§ 371 Date: Dec. 4, 2024

Related U.S. Application Data
(60) Provisional application No. 63/350,058, filed on Jun. 8, 2022.
(60) Provisional application No. 63/459,658, filed on Apr. 16, 2023.

(51) Int. Cl. A61K 40/11; A61K 40/46; A61P 31/20; C12N 5/0783

(52) U.S. Cl. CPC A61K 40/11; A61K 40/46; A61P 31/20; C12N 5/0636; C12N 2501/2302; C12N 2502/1121

(57) ABSTRACT

Provided herein are compositions comprising isolated T cells specific for one or more viral antigens derived from viruses such as adenovirus (ADV), cytomegalovirus (CMV), BK virus (BKV), Epstein-Barr virus (EBV), human herpes virus 6 (HHV6), John Cunningham virus (JC), or human immunodeficiency virus (HIV), methods for obtaining them, libraries including them, and uses thereof in treating viral infections.

### FIELD OF THE INVENTION

The present disclosure is generally directed to cell-based therapies for specifically targeting cells infected with viruses. In particular, the present disclosure is generally directed to compositions comprising isolated T cells directed against various viruses, methods for their preparation and uses thereof for treating viral infections in subjects.

### BACKGROUND OF THE INVENTION

Advanced therapies that expose patients to intensive antitumor chemotherapy, allogeneic hematopoietic stem cell transplantation (allo-HSCT), and solid organ transplantation (SOT) cause significant, life-threatening immune suppression. This immunocompromised state exposes patients to opportunistic, seasonal, and environmental infections, as well as viral reactivation that significantly increases morbidity and mortality rates and severely affects the outcome of alloHSCT and SOT. The ability to combat these threats mostly depends on post-transplant immune reconstitution which is a complex process that donor, recipient, and iatrogenic factors modulate. While innate immunity takes between 1 and 3 months to recover, adaptive immunity can take one year or more. In particular, the resulting B and T cell lymphopenia renders the transplant recipient vulnerable to opportunistic viral infections. These infections are the leading cause of transplant-related high morbidity and mortality rates. According to the European Society for Blood and Marrow Transplantation (EBMT), bacterial, fungal, and viral infection incidences may occur after auto-HSCT in 5-10% of patients, but with an increased rate of 20-50% following allo-HSCT, haplo-HSCT, and cord blood transplantation. The disease incidence increase in the pre-engraftment period is elongated according to the HLA matching status between donor and patient.

Conventional antiviral and antifungal pharmacotherapies have many limitations, including drug toxicity and the emergence of resistant infectious organisms. Antiviral agents such as ganciclovir, valganciclovir, cidofovir, and others have been shown to cause high rates of pancytopenia, neutropenia, and nephrotoxicity among bone marrow transplant patients. Antifungal agents such as amphotericin B, azoles, and echinocandins have severe adverse effects on renal and hepatic function. Moreover, the incidence of antiviral and antifungal resistance is rising, and chronic infection has been linked to T cell exhaustion and dysfunction.

Adoptive cell therapy explicitly directed against the pathogen target molecules is an emerging modality to effectively reduce or prevent the clinical manifestation of viral infections in immune-compromised patients. Donor-derived T cells targeting viral peptides were shown to be safe and effective. However, the personalized nature of this approach and the requirement for virus immune matched donors have emerged as barriers across the diversity of HLA alleles and the resulting number of potential genotypic combinations for under-represented ethnic and racial backgrounds patients.

Thus, there is a need in the art for local “off the shelf” biobank and more effective VSTs production methods to specifically target cells in a variety of individuals infected by a variety of viruses.

### SUMMARY OF INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The present invention relates to methods for preparing virus-specific T cells (VSTs) from allogeneic donors by activation with synthetic viral peptides derived from relevant viruses, such as adenovirus (ADV), cytomegalovirus (CMV), and BK virus (BKV), and the VSTs produced by these methods. These VSTs can further be expanded, functionally analyzed and cryopreserved for future therapeutic needs. One of the goals of the present invention is preserving a collection of such VSTs from different donors, having various HLA types, as a biobank.

According to some embodiments, there are provided herein compositions and methods to specifically target virally infected cells, and in particular, human cells infected with various types of viruses. In some embodiments, the compositions include isolated T cells capable of specifically recognizing viral antigens and which can consequently induce an appropriate immune response against virally infected cells, to thereby treat the viral infection. Further provided are methods of preparing such compositions and uses thereof.

In some embodiments, there is provided a composition comprising isolated T cells specific for one or more viral antigens derived from one or more viruses.

In some embodiments, at least 90% of the cells in the composition are T cells. In some embodiments, at least 60% of the cells in the composition are CD4+ T cells. In some embodiments, at most 30% of the cells in the composition are CD8+ T cells. In some embodiments, the CD4+ T cells and the CD8+ T cells in the composition are at a ratio of between about 2:1 and about 4:1.

In some embodiments, at least 40% of the cells in the composition are reactive T cells, specific for the one or more viral antigens. In some embodiments, at least 40% of the CD4+ T cells in the composition are reactive CD4+T cells, specific for the one or more viral antigens. In some embodiments, at most 20% of the CD8+ T cells in the composition are reactive CD8+ T cells, specific for the one or more viral antigens. In some embodiments, at least 30% of the cells in the composition are reactive CD4+ T cells.

In some embodiments, the one or more viruses are selected from: adenovirus (ADV), cytomegalovirus (CMV), BK virus (BKV), John Cunningham virus (JC), Epstein-Barr virus (EBV), human herpes virus 6 (HHV6), human immunodeficiency virus (HIV), and any combination thereof. In some embodiments, the one or more viruses comprise ADV and/or CMV. In some embodiments, the isolated T cells are specific for two or more, or three or more different viral antigens. In some embodiments, the one or more viral antigens are derived from two or more, or three or more, different viruses. In some embodiments, the three or more different viruses comprise ADV, CMV, and/or BKV.

In some embodiments, the isolated T cells specific for one or more viral antigens do not comprise T cells expressing a chimeric antigen receptor (CAR) or a recombinant T cell receptor (TCR).

In some embodiments, the compositions of the invention further comprise a pharmaceutically acceptable carrier.

In some embodiments, there is provided the composition of the invention for use in a method of treating a subject infected with a virus, or for preventing a viral infection in a subject at risk, the method comprising administering to the subject a therapeutically effective amount of the composition.

In some embodiments, the virus is selected from: ADV, CMV, BKV, EBV, HHV6, JC, and HIV.

In some embodiments, the administration is following transplantation of an organ or cells from a transplantation donor, such as a hematopoietic stem cell transplantation (HSCT) or a solid organ transplantation (SOT).

In some embodiments, the treating or preventing is conducted by adoptive cell therapy (ACT).

In some embodiments, the T cells are autologous or allogeneic to the subject. In some embodiments, the T cells are HLA-matched to or haploidentical with the subject. In some embodiments, the T cells are not derived from the subject or from the transplantation donor.

In some embodiments, there is provided a method for obtaining isolated T cells specific for one or more viral antigens derived from one or more viruses, the method comprising the steps of:
a) obtaining precursor cells from a biological sample of a donor;
b) preparing an in vitro stimulation (IVS) reaction by adding to the precursor cells (i) stimulating antigens derived from the one or more viral antigens, or (ii) antigen presenting cells (APCs) presenting peptides derived from the one or more viral antigens; and
c) incubating the IVS reaction of step (b) to obtain virus-specific T cells.

In some embodiments, the IVS is direct IVS, conducted by incubation of the precursor cells with the stimulating antigens. In some embodiments, the IVS is indirect IVS, conducted by incubation of the precursor cells with antigen presenting cells (APCs) presenting peptides derived from the one or more viral antigens.

In some embodiments, the method further comprises, prior to step (b), a step of incubating the APCs with the stimulating antigens to allow the APCs to present peptides derived from the one or more viral antigens.

In some embodiments, the incubating of the IVS reaction in step (c) is conducted for a length of about 8-15 days, or about 10-12 days.

In some embodiments, the IVS reaction comprises a priming step prior to the incubation in step (c), in which the IVS reaction is incubated for a length of about 1-5 hours, such as about 2 hours in a small volume, and the volume is increased following the priming step, in step (c).

In some embodiments, the small volume is about 0.5-10 ml, or about 2 ml.

In some embodiments, the volume is increased by about 10-500 fold, or by about 100-200-fold in step (c).

In some embodiments, IL2 is added to the IVS reaction during the incubation in step (c). In some embodiments, the IL2 is added after between about 2-4 days of incubation. In some embodiments, IL2 is the only cytokine added to the IVS reaction during the incubation. In some embodiments, IL2 is added to a final concentration of about 300 IU/ml. In some embodiments, no cytokine is added to the IVS reaction before between about 2-4 days of incubation have passed.

In some embodiments, the donor is not pre-treated with granulocyte colony stimulating factor (G-CSF) prior to donating the biological sample.

In some embodiments, the stimulating antigens comprise peptides having a length of about 12-18 amino acids.

In some embodiments, the stimulating antigens comprise peptides derived from the same antigen and which include an overlapping sequence of two or more amino acids.

In some embodiments, the method further comprises isolating and/or enriching for the virus-specific T cells obtained. In some embodiments, the method further comprises isolating and/or enriching for virus-specific CD4+ T cells from the obtained virus-specific T cells. In some embodiments, the method further comprises isolating and/or enriching for virus-specific CD8+ T cells from the obtained virus-specific T cells.

In some embodiments, the method further comprises a step of culturing the isolated T cells prior to preparing the IVS reaction in step (b).

In some embodiments, the one or more viruses are selected from: ADV, cytomegalovirus (CMV), BKV, JC, EBV, HHV6, HIV, and any combination thereof.

In some embodiments, there are provided isolated T cells specific for one or more viral antigens prepared by the method disclosed herein.

In some embodiments, there is provided a method of treating a disease caused by a virus, the method comprising administering a therapeutically effective amount of the compositions disclosed herein, or the isolated T cells disclosed herein, to a subject infected with the virus.

In some embodiments, there is provided a library comprising a plurality of compositions, each as disclosed herein, wherein the library comprises at least two compositions, in which: the isolated T cells in both compositions are specific for viral antigens derived from the same virus, but the viral antigens in one composition are restricted by a different HLA type than in the other composition.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1 is a schematic illustration of the preparation of compositions which include isolated T cells against viral antigens, according to some embodiments.

FIGS. 2A-2D show the results of in vitro stimulation of T cells with pathogen-specific peptides. PBMCs were in vitro stimulated with APCs loaded with a CEF HLA class I control peptide pool (CEFX™) twice (IVS1, FIG. 2C, and IVS2, FIG. 2D). Cells were restimulated and tested for the surface expression of 4-1BB using flow cytometry. APCs without peptide (no peptide, FIG. 2A) and non-stimulated PBMCs (No IVS, FIG. 2B) served as negative controls.

FIGS. 3A-3E show in vitro stimulation of T cells with adenovirus (ADV) specific peptide libraries. PBMC were in vitro stimulated with APCs loaded with the antigens: hexon, penton, or hexon+penton-specific peptide pools. Unstimulated and CEF stimulated PBMCs served as negative and positive controls, respectively. Cells were then restimulated with the above antigens or with PMA/ION-non-specific stimulation cocktail (positive control), and tested for the surface expression of OX40 and 4-1BB (CD3⁺4-1BB⁺OX40⁺ cells-checkered bars, left bar each pair) using flow cytometry and for secretion of IFNγ by ELISA (IFN, hatched bars, right bar each pair). Each graph shows the T cell populations enriched by a specific peptide library against: no antigen (FIG. 3A); CEF peptide pool (FIG. 3B), hexon (ADV) (FIG. 3C); penton (ADV) (FIG. 3D), and hexon+penton (ADV) (FIG. 3E).

FIG. 4 shows infection of donor-derived monocytes with different ADV subtypes. Donor PBMC were rested for two hours, and non-adherent cells were dispensed. ADV samples were added at different multiplicity of infection (MOI) levels. No. of ADV copies was measured at 24-48 hours post-infection using real-time PCR. Dark filled circles: control (no virus); light squares: ADV GFP MOI 1000; light triangles: ADV GFP MOI 3000; dark upside-down triangles: ADV5 MOI 0.001; dark diamonds: ADV3 MOI 0.5.

FIGS. 5A-5F show direct vs. indirect in vitro stimulation (IVS) with Hexon peptide library. FIG. 5A. Indirect (by APCs, upper panel) and direct (all PBMCs, lower panel) IVS schemes. FIGS. 5B-5F. Testing of the product of the direct/indirect IVS as well as no peptide control against donor PBMCs loaded with the indicated antigen: FIG. 5B. IVS with no peptide; FIG. 5C. Indirect IVS with a Hexon library; FIG. 5D. direct IVS with a Hexon library; FIG. 5E. indirect IVS with a CEF peptide pool; FIG. 5F. direct IVS with a CEF peptide pool. The T cells were stained for CD3 and for activation markers 4-1BB and OX40 and analyzed by FACS (checkered bars, left each pair), and the supernatant was analyzed for IFNγ level by ELISA (hatched bars, right each pair). PMA/ION-positive control.

FIGS. 6A-6F show single vs. triple IVS against adenovirus (ADV), BK virus (BKV), and cytomegalovirus (CMV). FIG. 6A. A scheme of the direct IVS process including T cell activation, expression of activation markers, and secretion of cytokines. FIGS. 6B-6F. Testing the IVS product by a co-culture with donor APCs loaded by the indicated antigen: FIG. 6B. IVS with no peptide. FIG. 6C; IVS with adenovirus peptides (Hexon library); FIG. 6D. IVS with BKV peptides VP1+LTA; FIG. 6E. IVS with CMV peptide (PP65); FIG. 6F. Triple IVS (peptides for ADV, BKV, and CMV). The T cells were stained for CD3 and for activation markers 4-1BB and OX40 and analyzed by FACS (checkered bars, left each pair), and the supernatant was analyzed for IFNγ level by ELISA (hatched bars, right each pair). Adenovirus peptides: Hexon; BKV peptides: VP1+LTA; CMV peptides: pp65; triple: Hexon+VP1+LTA+pp65. PMA/ION-positive control.

FIGS. 7A-7D show a functional co-culture validation of the virus-specific T cells (VSTs) against live ADV strains. FIG. 7A. The experimental scheme—top panel, donor PBMCs infected with ADV and reaction with VSTs; bottom panel—A549 cells PBMCs infected with ADV and reaction with VSTs. FIG. 7B. Propagation of ADV in dendritic cells vs. A549 following infection by the indicated virus/control. X shape: control A549 cells; empty upward triangle: ADV 3 in A549; filled circle: ADV 5 in A549; empty downward triangle: ADV GFP in A549; star: control dendritic cells (DV); asterisk: ADV 3 in DC; empty hexangle: ADV 5 in DC; filled diamond: ADV GFP in DC. FIG. 7C. Co-culture of ADV-infected A549 with trivalent VSTs. FIG. 7D. Co-culture of ADV-infected dendritic cells (DCs) with trivalent VSTs. The T cells were stained for CD3 and for activation markers 4-1BB and OX40 and analyzed by FACS (checkered bars, left each pair), and the supernatant was analyzed for IFNγ level by ELISA (hatched bars, right each pair). PMA/ION-positive control.

FIGS. 8A-8C show a large-scale, GMP-compliant IVS in a G-REX® production platform against 3 viruses (ADV, BKV, CMV). FIG. 8A. CD4⁺ vs. CD8⁺ compartment out of the total cells before and after the trivalent IVS. FIGS. 8B and 8C. Reactivity of the large-scale trivalent VST. The T cells were stained for CD3 and for activation markers 4-1BB and OX40 and analyzed by FACS (checkered bars, left each pair), and the supernatant was analyzed for IFNγ level by ELISA (hatched bars, right each pair). FIG. 8B. Total cells, and FIG. 8C. % reactive CD4⁺ T cells out of total CD4⁺ T cells (black) and % reactive CD8⁺ T cells out of total CD8⁺ T cells (hatched).

FIGS. 9A-9C describe VSTs used for treatment of a pediatric patient. FIG. 9A shows the reactivity of VSTs produced against ADV hexon and CMV PP65 libraries to antigens, as described above. FIGS. 9A-9B demonstrate the levels of viral load after bone marrow (B.M.) transplantation with infusion of VSTs. FIG. 9B. Treatment of ADV infection by administration of VSTs during weeks 6, 9, and 14. FIG. 9C. Treatment of CMV infection by administration of VSTs during weeks 6, 9, and 14.

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

The present invention provides pathogen-specific T cells and compositions including them for allogeneic use as antimicrobial therapies, such as antiviral, antibacterial or antifungal therapies.

The present invention further provides a system, or library comprising pathogen-specific T cells, thereby facilitating the production and banking of pathogen-specific T cells, or specifically of anti-virus-specific T cells. By utilizing pharmaceutical compositions which include these specific T cells, the immune system can consequently generate an immune response to clear away the viral infection.

According to some embodiments, the present invention advantageously provides isolated T cells specifically recognizing viral antigens, compositions comprising the same and methods of using the same for treating virally infected cells. Further provided are methods of preparing such isolated T cells. In some embodiments, the viral antigens are derived from various viruses, including, for example, but not limited to: ADV, BKV, EBV, CMV, HHV6, JC and/or HIV.

Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The term “a” and “an” refers to one or to more than one (i.e., to at least one) of the grammatical object of the article. By way of example, “an element” means one element or more than one element.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 90% and 110% of the given value.

For purposes of clarity, and in no way limiting the scope of the teachings, unless otherwise indicated, all numbers expressing quantities, percentages or proportions, and other numerical values recited herein, should be interpreted as being preceded in all instances by the term “about”, regardless of whether “about” is explicitly prepended to the numerical value. Accordingly, the numerical parameters recited in the present specification are approximations that may vary depending on the desired outcome. For example, each numerical parameter may be construed in light of the number of reported significant digits and by applying ordinary rounding techniques. Accordingly, unless indicated to the contrary, the numerical parameters set forth in this specification are approximations that may vary by up to plus or minus 10% depending upon the desired properties to be obtained by the present invention.

It is further clarified that for any list of values that is preceded by a phrase such as “at least”, “about”, or “at least about”, each value in the list is interpreted to also be preceded by the same phrase preceding the first value.

In the description and claims of the application, the words “include” and “have”, and forms thereof, are not limited to members in a list with which the words may be associated. As used herein, the term comprising includes the term consisting of.

The terms “peptide” and “protein” are used herein to refer to polymers of amino acid residues. Generally, “peptide” relates to a short polymer of amino acid residues (as detailed below), while “protein” generally relates to a complete protein. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the peptide or the protein can contain modifications, such as but be not limited only to, glycosylation, phosphorylation or disulfide bond shape.

As used herein, the term “in vitro” refers to an artificial environment and to processes or reactions that occur within an artificial environment. In vitro environments can consist of, but are not limited to, test tubes and cell culture. The term in vivo refers to the natural environment (e.g., an animal or a cell), and to processes or reactions that occur within a natural environment.

As used herein, the terms “subject”, “patient” or “individual” generally refer to a human, although the methods of the invention are not necessarily limited to humans and should be useful in other mammals.

As used herein, the term “donor” or “healthy donor” refers to a donor who provided a sample for preparing the VSTs of the invention. The donor may have provided the sample specifically for this purpose, or the donor may be a general donor, such as a blood donor, for which a sample was already available. In some embodiments, the donor is not infected with a respective virus. While blood donors must comply with certain health standards, it is not possible to know whether the donor or donors might have an undisclosed or unknown health condition.

The term “transplantation donor” refers specifically to a donor of transplanted organ or cells. In some embodiments, the donor is also the transplantation donor. In some embodiments, the donor is not the transplantation donor. In some embodiments, the donor is the subject who needs treatment.

The present invention is directed to the preparation of virus-specific T cells (VSTs) specific to a wide variety of viruses. The VSTs are useful for treating or preventing viral infections, especially viral infections that occur as a result of an organ transplantation. The VSTs are generally prepared by challenging T cells obtained from a donor with antigens derived from relevant viruses. Furthermore, the present invention is directed to VSTs prepared from individuals having different

HLA types, which are collected and stored as a library, for future use. This library, which includes HLA-versatile VSTs, becomes useful in providing a quick anti-viral treatment to a subject in need, since HLA-matched VSTs specific to the virus infecting the subject may be readily found in the library.

In some embodiments, the present invention provides a composition comprising isolated T cells specific for one or more target antigens derived from one or more target microorganisms.

In some embodiments, the target microorganisms is selected from a virus, a fungus, or a bacterium.

The description below relates mainly to target antigens which are viral antigens derived from viruses. However, the embodiments described below generally apply to other microorganisms, e.g., bacteria or fungi, mutatis mutandis.

In some embodiments, the present invention provides a composition comprising isolated T cells specific for one or more viral antigens derived from one or more viruses.

As used herein, the term “T cell” refers to any type of T cell, including cells expressing CD3 (CD3⁺), CD8 (CD8⁺), CD4 (CD4⁺), and/or other relevant T cells markers. In some embodiments, T cells express at least CD3.

The term “isolated T cells”, as used herein, is meant to clarify that the T cells are not part of the natural immune system of an individual, but rather are derived from a sample obtained from an individual, herein referred to as a donor. The term “isolated” is not intended to imply that the T cells are necessarily isolated, or separated from other cells originally present in the sample, or that the composition comprises only T cells. In some embodiments, the composition also comprises cells other than T cells, such as other immune cells. In some embodiments, the composition comprises mononuclear cells other than T cells, including but not limited to, monocytes, macrophages, and/or other antigen presenting cells (APCs), such as dendritic cells. In some embodiments, the composition comprises only T cells. In some embodiments, the composition comprises only T cells specific for one or more viral antigens derived from one or more viruses. According to some embodiments, at least 70%, 80%, 90%, 95%, 97% or 100% of the cells in the composition are T cells. According to some embodiments, about 100% of the cells in the composition are T cells. According to some embodiments, at least 95% of the cells in the composition are T cells.

According to some embodiments, at least 30%, 40%, 50%, or 60% of the cells in the composition are reactive T cells, specific to the one or more viral antigens. In some embodiments, at least 50% of the cells in the composition are reactive T cells. In some embodiments, about 40%-100%, 40%-90%, 50%-100%, 50%-90%, 60%-100%, or 60%-90%, of the cells in the composition are reactive T cells.

The terms “specific for” or “reactive to” used herein with respect to cells (e.g., T cells, CD3⁺ T cells, CD4⁺ T cells, CD8⁺ T cells, virus-specific cells) or TCRs, and an antigen (e.g., a viral antigen, a virus, a viral peptide), indicate the ability of the cells or TCRs to be activated by the respective antigen, and therefore to be able to elicit an immune response against the antigen. Stating that a cell is specific for an antigen also means that the cell expresses a TCR that is specific for the antigen. When activated by the respective antigen (via the TCR), the reactive (specific) cells express activation markers such as 4-1BB and/or OX40, and/or secrete INFγ. Reactivity of the reactive cells may be defined by various methods. In some embodiments, reactivity of cells is defined by their expression of activating markers, such as, e.g., as may be determined by a fluorescence cell sorter (FACS) analysis, or by any other suitable method. In some embodiments, reactivity of cells is defined by their INFγ expression or secretion, such as by a specified dye or agent that specifically labels INFγ expressing cells.

Accordingly, the phrase “reactive T cells”, as used herein, means: T cells specific (reactive) to the one or more viral antigens.

In some embodiments, the composition includes at least about 10⁶, 10⁷, 10⁸, 10⁹, or 10¹⁰ isolated T cells. For example, the composition may include from about 5×10⁵ to about 5×10⁶ T cells per ml, in a volume of from 50 to 200 ml. In some embodiments, the cells of the composition in various embodiments are at least 70% viable, and provided in a sterile medium, which may be a cryoprotectant medium (for example, 10% DMSO).

In some embodiments, T cells specific for the viral antigens further include T memory stem cells (Tscm). When administered to a subject, as described below with reference to methods of treatment, the composition therefore provides a durable response, including in vivo persistence of antigen-specific T cells for at least about 1 month, or at least about 3 months, or at least about 6 months, or at least about 12 months, or at least about 18 months, or at least about two years in some embodiments.

While a viral response is typically a cytotoxic reaction, involving CD8⁺ T cells, the inventors have found that a high level of CD4⁺ T cells is also required.

According to some embodiments, at least 40%, 50%, 60%, 70%, 80% or 90% of the cells in the composition are CD4⁺ T cells. According to some embodiments, at least about 60%, 70%, or 80% of the cells in the composition are CD4⁺ T cells. According to some embodiments, about 100% of the cells in the composition are CD4⁺ T cells. According to some embodiments, about 50%-100%, 50%-90%, 60%-100%, 60%-90%, 70%-100%, or 70%-90%, of the cells in the composition are CD4⁺ T cells.

According to some embodiments, at most 20%, 30%, 40%, or 50% of the cells in the composition are CD8⁺ T cells. According to some embodiments, at most 30% or 40% of the cells in the composition are CD8⁺ T cells. According to some embodiments, about 1%-20%, 5%-20%, 1%-30%, 5%-30%, 1%-40%, 5%-40%, 1%-50%, or 5%-50%, of the cells in the composition are CD8⁺ T cells.

In some embodiments, the cell composition may include at least about 10⁷, 10⁸, 10⁹, or 10¹⁰ CD4⁺ T cells. For example, the composition may include from about 5×10⁵ to about 5×10⁶ CD4⁺ T cells per ml, in a volume of from 50 to 200 ml.

According to some embodiments, the composition comprises about 50% to about 80% CD4⁺ T cells and about 20% to about 40% CD8⁺ T cells out of the total T cells. According to some embodiments, the composition comprises about 60% to about 75% CD4⁺ T cells and about 30% to about 40% CD8⁺ T cells out of the total T cells. According to some embodiments, the composition comprises at least 60%, 70%, or 80% CD4⁺ T cells, and at most 20%, 30%, or 40% CD8⁺ T cells out of the total T cells. According to some embodiments, the composition comprises at least 60% CD4⁺ T cells, and at most 30% CD8⁺ T cells out of the total T cells. According to some embodiments, the composition comprises at least 70% CD4⁺ T cells, and at most 25% CD8⁺ T cells out of the total T cells.

According to some embodiments, the CD4⁺ T cells and the CD8⁺ T cells in the composition are at a ratio of between about 2:1 and about 4:1 CD4⁺ T cells to CD8⁺ T cells.

According to some embodiments, at least 40% or 50% of the CD4⁺ T cells are reactive CD4⁺ T cells. According to some embodiments, about 40%-100%, 40%-90%, 50%-100%, or 50%-90%, of the CD4⁺ T cells are reactive CD4⁺ T cells.

According to some embodiments, at most 10% or 20% of the CD8⁺ T cells are reactive CD8⁺ T cells. According to some embodiments, about 1%-20%, 1%-10%, or 1%-5%, of the CD8⁺ T cells are reactive CD8⁺ T cells.

Since about 70% or more of the cells in the composition are CD4⁺ T cells, then according to some embodiments, at least 20%, 25%, 30%, 35%, or 40%, of the cells in the composition are reactive CD4⁺ T cells, specific to the one or more viral antigens. According to some embodiments, at least 30%, 35%, or 40%, of the cells in the composition are reactive CD4⁺ T cells. According to some embodiments, about 20%-90%, 25%-90%, 30%-90%, 35%-90%, 40%-90%, 20%-80%, 25%-80%, 30%-80%, 35%-80%, 40%-80%, 20%-70%, 25%-70%, 30%-70%, 35%-70%, 40%-70%, 20%-60%, 25%-60%, 30%-60%, 35%-60%, or 40%-60%, of the cells in the composition are reactive CD4⁺ T cells.

Since about 25% or less of the cells in the composition are CD8⁺ T cells, then according to some embodiments, at most 3% or 5% of the cells in the composition are reactive CD8⁺ T cells, specific to the one or more viral antigens. According to some embodiments, at most 3%, 5%, or 10% of the cells in the composition are reactive CD8⁺ T cells, specific for the one or more viral antigens. According to some embodiments, about 1%-10%, 1%-5%, or 1%-3% are reactive CD8+ T cells.

Accordingly, the ratio between reactive CD4⁺ T cells and reactive CD8⁺ T cells in the composition is at least about 10:1, 7:1, or 5:1.

As used herein, the term “virus” refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells.

Adenovirus (ADV), Cytomegalovirus (CMV), and BK virus (BKV) are three viruses which are commonly reactivated in pediatric patients following bone marrow transplantations. While no effective treatment is available for ADV and BKV infection, an effective treatment is available for CMV infections. ADV causes a life-threatening infection to immunocompromised patients, and BKV infection causes a high risk for organ rejection and repetitive hospitalization.

As used herein, the terms “Adenovirus” and “ADV” are directed to members of the family Adenoviridae, which are medium-sized (90-100 nm), nonenveloped viruses with an icosahedral nucleocapsid containing a linear, non-segmented double stranded DNA genome (size of about 26-46 Kbp). Over 50 serotypes of ADV are known. ADV may cause respiratory, intestinal, and eye infections.

As used herein, the term “Cytomegalovirus” or “CMV” is directed to the genus of viruses in the order Herpesvirales, in the family Herpesviridae, in the subfamily Betaherpesvirinae. The 11species in this genus include human betaherpesvirus 5 (HCMV, human cytomegalovirus, HHV-5), which is the species that infects humans. Diseases associated with HHV-5 include mononucleosis and pneumonia, and congenital CMV in infants can lead to deafness and ambulatory problems.

As used herein, the term “BKV” is directed to a BK virus of the polyomavirus family. In some embodiments, the one or more viruses are selected from: ADV, CMV, BKV, John Cunningham virus (JC), Epstein-Barr virus (EBV), human herpesvirus 6 (HHV6), human immunodeficiency virus (HIV), and any combination thereof.

In some embodiments, the one or more viruses comprise ADV. In some embodiments, the one or more viruses comprise CMV. In some embodiments, the one or more viruses comprise BKV.

In some embodiments, the one or more viruses are ADV. In some embodiments, the one or more viruses are CMV. In some embodiments, the one or more viruses are BKV.

In some embodiments, the one or more viruses comprise ADV and/or CMV. In some embodiments, the one or more viruses comprise ADV and/or BKV. In some embodiments, the one or more viruses comprise CMV and/or BKV. In some embodiments, the one or more viruses comprise ADV and CMV. In some embodiments, the one or more viruses comprise ADV and BKV. In some embodiments, the one or more viruses comprise CMV and BKV.

In some embodiments, the one or more viruses are ADV and CMV. In some embodiments, the one or more viruses are ADV and BKV. In some embodiments, the one or more viruses are CMV and BKV.

In some embodiments, the one or more viruses comprise ADV, CMV, and/or BKV. In some embodiments, the one or more viruses comprise ADV, CMV, and BKV. In some embodiments, the one or more viruses are ADV, CMV, and BKV.

The isolated T cells specific for one or more viral antigens of the present invention are T cells which are specific for viral antigens or for viral peptides or epitopes derived from the viral antigens, and are also referred to herein as virus-specific T cells (VSTs). In some embodiments, the VSTs have T cell receptors (TCR) which are specific for the viral antigens, and therefore the viral antigens (or peptides derived therefrom) may be recognized by the VSTs through their specific TCRs, thereby causing activation of the VSTs.

As used herein, the term “virus-specific T cell” or “VST” is directed to a T cell which can specifically recognize one or more viral antigens or peptides or epitopes thereof, and can further induce an immunogenic reaction against a viral particle or a cell expressing such antigens (i.e., an infected cell).

In some embodiments, the composition comprises VSTs specific for a single viral antigen. In some embodiments, the composition comprises VSTs specific for more than one viral antigen. In some embodiments, the composition comprises VSTs specific for more than two, or more than three different viral antigens. In some embodiments, the composition comprises VSTs specific for about 2-15, 5-15 or 5-10 viral antigens.

VST specificity toward a viral antigen may be determined by re-stimulation by the viral antigen (as described in the examples), or by MHC multimer staining (e.g., dimer or tetramer staining).

In some embodiments, the composition comprises VSTs specific for antigens derived from a single virus. In some embodiments, the composition comprises VSTs specific for antigens derived from more than one virus. In some embodiments, the composition comprises VSTs specific for antigens derived from two or more, or three or more different viruses. In some embodiments, the composition comprises VSTs specific for at least one antigen derived from one or more viruses selected from ADV, CMV, BKV, JC, EBV, HHV6, and HIV. In some embodiments, the composition comprises VSTs specific for more than one antigen derived from more than one virus selected from ADV, CMV, BKV, JC, EBV, HHV6, and HIV. In some embodiments, the composition comprises VSTs specific for two or more antigens or three or more antigens derived from two or more or three or more viruses selected from ADV, CMV, BKV, JC, EBV, HHV6, and HIV.

In some embodiments, the composition comprises VSTs specific for one or more ADV antigens. In some embodiments, the virus-specific T cell is capable of recognizing an antigen encoded by, expressed by, or derived from ADV genome. In some embodiments, the composition comprises VSTs specific for one or more CMV antigens. In some embodiments, the virus-specific T cell is capable of recognizing an antigen encoded by, expressed by, or derived from CMV genome. In some embodiments, the composition comprises VSTs specific for one or more BKV antigens. In some embodiments, the virus-specific T cell is capable of recognizing an antigen encoded by, expressed by, or derived from BKV Genome. In some embodiments, the composition comprises VSTs specific for one or more JC antigens. In some embodiments, the virus-specific T cell is capable of recognizing an antigen encoded by, expressed by, or derived from JC genome.

In some embodiments, the composition comprises VSTs specific for one or more EBV antigens. In some embodiments, the virus-specific T cell is capable of recognizing an antigen encoded by, expressed by, or derived from EBV genome. In some embodiments, the composition comprises VSTs specific for one or more HHV6 antigens. In some embodiments, the virus-specific T cell is capable of recognizing an antigen encoded by, expressed by, or derived from HHV6 genome. In some embodiments, the composition comprises VSTs specific for one or more HIV antigens. In some embodiments, the virus-specific T cell is capable of recognizing an antigen encoded by, expressed by, or derived from HIV genome.

As used herein, the term “viral antigen” refers to antigens which are, or are derived from, viral proteins. The viral antigen may be a complete protein or a partial protein, or a peptide derived from a viral protein.

In order to enrich for a population of T cells specific for certain viral antigens, stimulating antigens which are derived from the viral antigens, are presented to white blood cells from a donor, as described below, in order to cause activation and expansion of T cell clones from the white blood cells, which recognize the viral antigens. As explained in more detail below, the stimulating antigens may be the viral antigens themselves, portions of the viral antigens, peptides derived from the viral antigens, or relevant variants of the viral antigens.

The stimulating antigens may be presented directly to the donor cells, or they may be first presented to antigen presenting cells (APC) s or professional antigen presenting cells (pAPC, e.g., dendritic cells), which are then used for incubating with the donor cells.

According to the present invention, when T cells (or VSTs) are said to be specific for certain viral peptides which are derived from certain viral antigens, these T cells (or VSTs) are also meant to be specific for the viral antigens.

In some embodiments, the viral antigens are derived from one or more viruses selected from ADV, CMV, BKV, JC, EBV, HHV6, and HIV. In some embodiments, the viral antigens are derived from viral proteins encoded by ADV, CMV, BKV, JC, EBV, HHV6, and/or HIV genome.

In some embodiments, the viral antigens are selected structural proteins, functional proteins, extracellular proteins and/or intracellular proteins.

In some embodiments, the viral antigens are selected from the proteins hexon, penton, pp65, large T-antigen (LTA), and viral protein 1 (VP1). In some embodiments, the viral antigens are derived from the protein(s) hexon, penton, pp65, LTA, and/or VP1. In some embodiments, the viral antigens are, or are derived from, a hexon protein. In some embodiments, the viral antigens are, or are derived from, a penton protein. In some embodiments, the viral antigens are, or are derived from, a pp65 protein. In some embodiments, the viral antigens are, or are derived from, an LTA protein. In some embodiments, the viral antigens are, or are derived from, a VP1 protein. In some embodiments, the hexon protein is an ADV hexon protein. In some embodiments, the penton protein is an ADV penton protein. In some embodiments, the pp65 protein is a CMV pp65 protein. In some embodiments, the LTA protein is a BKV LTA protein. In some embodiments, the VP1 protein is a BKV VP1 protein.

Viral peptides are discussed in more detail below, with reference to the methods of preparing VSTs.

In some embodiments, the isolated T cells specific for one or more viral antigens do not comprise T cells expressing a chimeric antigen receptor (CAR) or a recombinant T cell receptor (TCR).

In some embodiments, when activated, the VSTs express on their surface activating markers including 4-1BB and OX40, and secrete cytokines including IL-2, IFNβ, and TNFα. Accordingly, measuring expression of 4-1BB and OX40 by FACS analysis, or measuring the secreted factors such as IFNγ by, e.g., ELISA, can indicate the level of activation of the VSTs.

In some embodiments, the cell composition includes T cells positive for activation markers selected from 4-1BB and OX40. In some embodiments, the cell composition includes CD3⁺CD4⁺4-1BB⁺OX40⁺ T cells and/or CD3⁺CD8⁺4-1BB⁺OX40⁺ T cells. In some embodiments, at least a portion of the specific T cells in the composition are CD3⁺CD4⁺4-1BB⁺OX40⁺ T cells and/or CD3⁺CD8⁺⁴-1BB⁺OX40⁺ T cells. In some embodiments, at least a portion of the specific T cells in the composition express IFNγ, TNFα and/or PD1. Each possibility is a separate embodiment.

In some embodiments, the compositions of the invention comprise cells of a desired or a pre-defined HLA type, such as an HLA-type in need.

In some embodiments, the compositions, and the isolated T cells (the VSTs), described herein above are prepared by methods of the invention, described herein below. Accordingly, compositions prepared by the below methods are also specifically covered by the present invention.

In some embodiments, the present invention provides a composition comprising isolated T cells specific for one or more viral antigens derived from one or more viruses, wherein the composition comprises at least 40%, 50%, or 60% isolated T cells specific for one or more viral antigens out of the total cells of the composition.

In some embodiments, the present invention provides a composition comprising isolated T cells specific for one or more viral antigens derived from one or more viruses, wherein the composition comprises at least 60% or 70% CD4⁺ T cells.

In some embodiments, the present invention provides a composition comprising isolated T cells specific for one or more viral antigens derived from one or more viruses, wherein the composition comprises at least 30% or 40% isolated CD4⁺ T cells specific for one or more viral antigens out of the total cells of the composition.

In some embodiments, the present invention provides the composition described herein, further comprising a pharmaceutically acceptable carrier.

In some embodiments, the pharmaceutical composition is prepared for immediate use in a patient in need thereof. In some embodiments, the pharmaceutical composition is prepared and stored for future use.

In some embodiments, the pharmaceutically acceptable carrier is a buffer, diluent, adjuvant, excipient, or vehicle suitable for administration with the VSTs. In some embodiments, the pharmaceutically acceptable carrier may be suitable for intravenous infusion. In some embodiments, the pharmaceutically acceptable carrier may be suitable as a cryoprotectant. In some exemplary embodiments, the carrier may be DMSO (for example, at about 10%). In some embodiments, the pharmaceutically acceptable carrier may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

In some embodiments, the compositions of the invention may be provided in unit vials or bags and stored frozen until use. Unit doses may include from about 5×10⁴ to about 5×10⁹ cells per ml, in a volume of from 50 to 200 ml. Each possibility is a separate embodiment.

In some embodiments, the present invention provides the composition described herein for administering to a subject in need thereof. The administration may be for treating a viral infection in the subject, or for preventing a viral infection in a subject at risk for developing it.

In some embodiments, the present invention provides the composition described herein for use in a method of treating a subject infected with a virus, the method comprising administering to the subject a therapeutically effective amount of the composition. In some embodiments, the present invention provides the composition described herein for use in a method of preventing a viral infection in a subject at risk, the method comprising administering to the subject a therapeutically effective amount of the composition.

In some embodiments, the present invention provides a composition comprising isolated T cells specific for one or more viral antigens derived from one or more viruses, for use in a method of treating a subject infected with a virus, the method comprising administering to the subject a therapeutically effective amount of the composition. In some embodiments, the present invention provides a composition comprising isolated T cells specific for one or more viral antigens derived from one or more viruses for use in a method of preventing a viral infection in a subject at risk, the method comprising administering to the subject a therapeutically effective amount of the composition.

In some embodiments, the present invention provides a use of the composition described herein for treating a subject infected with a virus, comprising administering to the subject a therapeutically effective amount of the composition. In some embodiments, the present invention provides a use of the composition described herein for preventing a viral infection in a subject at risk, comprising administering to the subject a therapeutically effective amount of the composition.

In some embodiments, the present invention provides a use of the composition described herein for the preparation of a medicament for treating a subject infected with a virus. In some embodiments, the present invention provides a use of the composition described herein for the preparation of a medicament for preventing a viral infection in a subject at risk.

The term “subject at risk”, or “subject at risk of being infected by a virus”, as used herein, relates to a subject who does not show symptoms of being affected with a virus, but is at risk for developing a viral disease. For example, this may be a subject who underwent organ transplantation (or some other procedure involving contact with human biological material), and it was later found that the transplantation donor was infected with a certain virus. The subject at risk may also be at risk because of a potential viral contamination in the surrounding of the subject, for example, by being in contact, or around an individual who was infected with a virus. When a subject is at risk for a known virus or viruses, administering the compositions or the VSTs of the invention may prevent a viral infection.

It is noted that the composition mentioned here with respect to its use is the same composition described herein above in more detail, and therefore all embodiments described above with respect to the composition also apply to the composition described here with respect to its use.

In some embodiments, the virus is selected from: adenovirus (ADV), cytomegalovirus (CMV), BK virus (BKV), John Cunningham virus (JC), Epstein-Barr virus (EBV), human herpes virus 6 (HHV6), and human immunodeficiency virus (HIV), and any combinations thereof.

In some embodiments, the administration is following transplantation of an organ or cells from a transplantation donor, such as a hematopoietic stem cell transplantation (HSCT) or a solid organ transplantation (SOT).

In some embodiments, the administration is part of an adoptive cell therapy (ACT). In some embodiments, the composition is for use in ACT against virally infected cells. As used herein, the terms “adoptive cell transfer therapy” or “ACT” refer to administration of ex vivo-activated and/or-expanded autologous or allogeneic viral-reactive T cells, either as is or in a suitable composition in the presence of one or more suitable excipients (such as, for example, a suitable buffer).

In some embodiments, the T cells are autologous or allogeneic to the subject. In some embodiments, the T cells are autologous to the subject. In some embodiments, the T cells are allogeneic to the subject. In some embodiments, the T cells are HLA-matched to or haploidentical with the subject.

The terms “autologous”, “allogeneic”, and “haploidentical”, as used herein, refer to the level of identity (match) between HLA molecules of donor (or transplantation donor) cells and of recipient cells.

The major histocompatibility complex (MHC) is a collection of cell surface molecules encoded by a large number of genes in mammals, which are extremely polymorphic and therefore variable in the population. MHC molecules, also referred to in humans as human leukocyte antigens (HLA), include Class I and Class II molecules. Class I molecules are expressed on all nucleated cells and present processed peptides from within the cell mainly to cytotoxic (CD8) cells, while class II molecules are expressed on the surface of immune system cells, and present external antigens mainly to CD4 cells.

HLA molecules are important for organ transplantations, and mismatched HLA types (i.e. transplantation donor and recipient expressing different HLA molecules) are a major cause of transplanted organs rejection. Therefore, the compatibility of an organ donation is determined, inter alia, by the level of identity of HLA molecules (HLA match) between the transplantation donor and the subject.

The term “autologous”, as used herein, refers to cells which are derived from the same subject and therefore have the same HLA type.

The term “allogeneic”, as used herein, refers to a donor, or transplantation donor, of the same species as the subject, but expressing HLA molecules at least to some extent different than those of the subject. Allogeneic cells may cause graft-host disease when used for cell or organ transplantation.

The term “haploidentical”, as used herein, refers to an allogeneic match, which is usually from a family member who is about 50% identical to the subject.

In some embodiments, the T cells are not derived from the subject or the transplantation donor. In some embodiments, the T cells are not derived from the subject. In some embodiments, the T cells are not derived from the transplantation donor.

In some embodiments, the present invention provides methods for obtaining virus-specific T cells. To this end, the cells are primed by culturing in the presence of stimulating antigens, which are derived from the viral antigens, or with antigen presenting cells (APCs) displaying viral peptides derived from the viral antigens. The APCs may display viral peptides derived from the viral antigens following incubation with the stimulating antigens, but it is also conceivable that the APCs include nucleic acids encoding the viral antigens or peptides derived therefrom and therefore display them on their surface.

In order to test for the activation of virus-specific T cells (VSTs), the primed cells are then re-stimulated and tested for their post priming cytokine production (for example, INF-y, IL-1, TNF-a, and the like), or for expression of activating markers such as 4-1BB and/or OX-40. In some embodiments, INF-γ producing cells or the 4-1BB and/or OX-40 expressing cells may be enriched by using suitable protocols (such as, for example, the Miltenyi Biotec pre-clinical INF-γ enrichment procedure). The enriched cells may then be rapidly expanded. In some embodiments, the obtained cells may then be tested for their cytotoxic activity (i.e., killing) of virally infected cell-lines (cells infected with the virus or with viral antigens) and/or against donor cells loaded with specific viral antigens. The identified specific and active T cells may further be used in clinical applications, by administration in a suitable form (composition) to subjects infected with a virus. In some embodiments, the cells may be cryopreserved for further use.

Accordingly, in some embodiments, the present invention provides a method for obtaining isolated T cells specific for one or more viral antigens derived from one or more viruses, the method comprising the steps of:

a) obtaining precursor cells from a biological sample of a donor;

b) preparing an in vitro stimulation (IVS) reaction by adding to the precursor cells (i) stimulating antigens derived from the one or more viral antigens, or (ii) APCs presenting peptides derived from the one or more viral antigens; and c) incubating the IVS reaction of step (b) to obtain virus-specific T cells (VSTs).

The term “precursor cells” as used here, relates to donor cells which are suitable for the invention. The precursor cells must include T cells, and therefore may be any group of cells including T cells, including, but not limited to white blood cells, peripheral blood mononuclear cells (PBMCs), mononuclear cells (MNCs), immune cells, and T cells.

The precursor cells may be obtained from a fresh or from a frozen biological sample. In some embodiments, the precursor cells are cultured cells.

In some embodiments, the precursor cells are PBMCs.

The biological sample may be any biological sample which includes precursor cells (and specifically T cells), as defined above. Suitable biological samples include, but are not limited to a blood sample, a blood product sample containing T cells, a fraction of a blood sample containing T cells (such as a buffy coat fraction), a bone marrow tissue sample, a lymph node tissue sample, and a spleen tissue sample.

In some embodiments, the biological sample is a blood sample. In some embodiments, the biological sample is a blood sample donated to a blood bank. In some embodiments, the biological sample is a blood product containing T cells. In some embodiments, the biological sample is a blood product containing PBMCs. In some embodiments, the biological sample is a fraction of a blood sample, which contains T cells, such as a buffy coat.

The term “buffy coat” relates to the fraction of an anticoagulated blood sample which is at the interface between the red blood cells fraction and the plasma, and contains most of the white blood cells and platelets following centrifugation, when blood is taken with an anticoagulant, preferably a divalent ion chelator such as EDTA.

In some embodiments, precursor cells are obtained from the biological sample by any suitable method known in the art. In some embodiments, precursor cells are obtained from a unit of blood collected from a donor using any number of techniques known to one or skill in the art. For example, precursor cells from the circulating blood of an individual can be obtained by apheresis or leukapheresis. The apheresis product typically contains lymphocytes, including T cells and precursor T cells, monocytes, granulocytes, B cells, other nucleated white blood cells, red blood cells, and platelets. Leukapheresis is a laboratory procedure in which white blood cells are separated from a sample of blood. Cells collected by apheresis can be washed to remove the plasma fraction and to place the cells in an appropriate buffer or media for subsequent processing steps. Washing steps can be accomplished by methods known to those in the art, such as by using a semi-automated “flow-through” centrifuge. After washing, the cells may be resuspended in a variety of biocompatible buffers, such as, for example, Ca-free, Mg-free PBS. Alternatively, the undesirable components of the apheresis sample can be removed, and the cells directly re-suspended in a culture medium. If desired, precursor cells can be isolated from peripheral blood lymphocytes by lysing the red blood cells and depleting the monocytes, for example, by centrifugation through a Ficoll® or a Percoll® gradient.

In some embodiments, the biological sample or the precursor cells are tested for determination of the donor HLA type. HLA typing of the donor sample is performed for the purpose of future determination of at least partial matching between the donor cells and a potential recipient. In some embodiments, the HLA typing is performed based on the population repertoire (see, for example, the Allele frequency net database (AFND)). However, HLA typing may be performed by any suitable method (e.g., by serology or by DNA typing), and may take place at any time during processing of the sample or the preparation of the VSTs.

In some embodiments, the donor has a desired (or pre-defined) HLA-type. In some embodiments, the donor is autologous to a specific subject. In some embodiments, the donor is allogeneic to a specific subject. In some embodiments, the donor is haploidentical to a specific subject.

It is noted that bone marrow transplantation (BMT) transplantation donor donors are usually treated prior to the donation of the sample by stimulating their bone marrow to produce and mobilize stem cells. This may be done, e.g., by administration of granulocyte-colony stimulating factor, G-CSF, or similar factors, prior to donation.

According to the present invention, such bone marrow stimulation and mobilization of stem cells is not necessary, and the inventors even have a reason to believe that such treatment exhausts the stem cells and makes them less active. Accordingly, in some embodiments, the donor is not pre-treated (e.g. by G-CSF) to stimulate bone marrow and to induce mobilization of stem cells prior to donation. The importance of the lack of need for pretreatment is that any blood bank sample may be used according to the invention, which highly increases the availability of a wide variety of samples which may be suitable for use in the method of the invention.

Nevertheless, in some embodiments, the donor may be pre-treated by G-CSF or similar agents.

In some embodiments, the precursor cells are first preincubated, before mixing with the stimulating antigens derived from the one or more viral antigens in step (b). The preincubation may be around 1-3 h, or about 2 h. In some embodiments, the precursor cells are not preincubated prior to mixing.

In step (b) of the method, the stimulating antigens derived from the one or more viral antigens, or APCs presenting peptides derived from the viral antigens, are added to the precursor cells for IVS.

In some embodiments, the APCs are professional APCs, such as dendritic cells, B cells, or macrophages, which can present an antigen both in the context of class I HLA molecules and class II HLA molecules. In some embodiments, the APCs are any nucleated cell, which presents antigens in the context of class I HLA molecules. In some embodiments, the APCs are professional APCs. In some embodiments, the APCs are dendritic cells or macrophages. The stimulating antigens derived from the one or more viral antigens are added in order to activate and expand T cell clones which are capable of recognizing the viral antigens. Accordingly, the stimulating antigens derived from the one or more viral antigens may be provided in any suitable form for stimulating T cell clones which recognize the viral antigens. The stimulating antigens derived from the one or more viral antigens may be the complete viral antigens, portions of the viral antigens, or peptides derived from the viral antigens, that are capable of eliciting a response against the viral antigens. It is also conceivable that the stimulating antigens will include modifications or variations compared to the viral antigens.

The peptides derived from the viral antigens may cover the whole antigen, or only certain regions thereof, such as the immunogenic regions thereof, such as the epitopes. The stimulating antigens may correspond to a single viral protein, several proteins, or all viral proteins of the virus. The peptides derived from the viral antigens may be a single specific peptide derived, e.g., from an epitope of a viral protein, or may be a collection, or a library or peptides derived from one viral protein, or from more than one viral protein.

In some embodiments, the stimulating antigens are pools of peptides derived from at least one viral antigen. In some embodiments, the stimulating antigens are pools of peptides derived from an ADV hexon protein. In some embodiments, the stimulating antigens are pools of peptides derived from a CMV pp65 protein. In some embodiments, the stimulating antigens are pools of peptides derived from BKV VP1 and/or LTA.

It is noted that for APC loading, the stimulating antigens may be used, and the APCs will present peptides derived from the viral antigens based on their HLA type and cellular processing.

In some embodiments, the stimulating antigens are proteins derived from one or more viruses. In some embodiments, the stimulating antigens are peptides derived from the viral antigens. In some embodiments, the peptides derived from the viral antigens cover the complete viral antigen they are derived from. In some embodiments, the peptides derived from the viral antigens cover only a portion of the viral antigen they are derived from. In some embodiments, the peptides derived from the viral antigens cover only epitope(s) derived from the viral antigen.

In some embodiments, the length of the peptides is between about 10-20, about 12-18, or about 15, amino acids. In some embodiments, the peptides derived from the viral antigens include at least two peptides derived from the same viral antigen, these at least two peptides having an overlap in sequence between them. In some embodiments, the overlap in sequence is at least 2-10 amino acids. In some embodiments, the overlap in sequence is at least 2, 3, 3, 4, 6, 8, or 10 amino acids. In some embodiments, any of the amino acid sequences of the stimulating antigens may include one or more modified amino acids.

In some embodiments, the stimulating antigens are generally suitable for presentation by an HLA-A, B, or C molecular complex, and in some embodiments an HLA-A2 molecular complex. In some embodiments, the stimulating antigens are generally suitable for presentation by HLA-DR/DP/DQ (Class II MHC) HLA complexes.

In some embodiments, a GMP (good manufacturing practice) or GLP (good laboratory practice) grade viral peptide library is used to prime the T cells. In some embodiments, the peptides derived from the viral antigens are from a commercial peptide library such as CEFX™M, a Hexon (ADV), a Penton (ADV), a PP65 (CMV) etc., available from Miltenyi Biotec.

When a collection, or a library of peptides is used for IVS (derived from a single viral antigen or from several viral antigens), then clones recognizing the different peptides are activated and become expanded. Therefore, VSTs specific to a variety of peptides will be obtained as a result.

Accordingly, in order to obtain a library of VSTs specific to a viral antigen or a virus, the stimulating antigens used for the IVS are derived from the viral antigen, or from antigens derived from the virus. However, in order to obtain a collection of VSTs specific to many viral antigens or to many viruses, the stimulating antigens used for the IVS are derived from a variety of viral antigens derived from all of the desired viruses.

Similarly, IVS conducted with APCs presenting peptides derived from a single virus or viral antigen will yield VSTs specific to the virus or viral antigen, and IVS conducted with APCs presenting peptides derived from a variety of viruses or viral antigens will yield VSTs specific to the variety of viruses or viral antigens.

According to some embodiments, the stimulating antigens may be added in step (b) in either of two ways, as explained below. According to some embodiments, stimulating antigens are mixed directly with the precursor cells, and the IVS reaction is then incubated for a “direct” IVS. According to some embodiments, stimulating antigens are mixed with APCs and incubated for the APCs to present peptides derived from the viral antigens. Then, the “loaded” APCs are incubated with the precursor cells for “indirect” IVS.

In some embodiments, “direct” IVS is used (option (i)) and the precursor cells comprise cells other than T cells. In some embodiments, “direct” IVS is used and the precursor cells comprise APCs. In some embodiments, “direct” IVS is used and the precursor cells comprise professional APCs. In some embodiments, the “direct” IVS is used and the precursor cells are PBMCs.

The APCs may be derived from the donor sample, or may be from a different source.

Therefore, according to some embodiments, the method further comprises, prior to step (b), a step of incubating the APCs with the stimulating antigens to allow the APCs to present peptides derived from the one or more viral antigens.

In step (c), the IVS reaction is incubated in order for the T cell clones which recognize the viral antigens to become activated and then for the activated clones to become expanded.

The IVS reaction may be incubated in any suitable medium for culturing monocytes or T cells, such as RPMI, AIM-VTM, or the VST medium described in the examples.

In some embodiments, the incubating of the IVS reaction is conducted for a length of about 8-15 days, or about 10-12 days.

In some embodiments, after preparing the IVS reaction in step (b) and prior to incubating in step (c) the method comprises a “priming step”, comprising incubating the IVS reaction in a small volume and for a relatively short time (up to several hours), for a more efficient IVS.

Accordingly, in some embodiments, the method further comprises a priming step prior to the incubation in step (c), in which the IVS reaction is incubated for a short length of time in a small volume (volume of the IVS reaction, or volume of the medium containing the IVS reaction), and the volume is increased following the priming step, in step (c).

In some embodiments, the short length of time is about 30 mins to about 5 hours, about 1 to about 5 hours, or about 2 hours.

In some embodiments, the small volume is about 0.5 ml to about 10 ml, about 1 ml to about 5 ml, or about 2 ml.

In some embodiments, the volume is increased in step (c) by about 10-fold to about 500-fold, about 50-fold to about 300-fold, or about 100-fold to about 200-fold.

In some embodiments, the volume is increased in step (c) to about 50 ml to about 1000 ml, about 100 ml to about 500 ml, about 200 ml to about 500 ml, or about 300 ml.

In some embodiments, a cytokine is added to the IVS reaction during the incubation. In some embodiments, a cytokine is added to the medium during the incubation.

In some embodiments, the cytokine is added to the IVS reaction after at least 2, 3, or 4 days of incubation. In some embodiments, the cytokine is added after between about 2-4 days of incubation.

In some embodiments, no cytokine is added to the IVS reaction before 2, 3, or 4 days of incubation have passed. In some embodiments, no cytokine is added to the IVS reaction before 2 days of incubation have passed. In some embodiments, no cytokine is added to the IVS reaction before 3 days of incubation have passed. In some embodiments, no cytokine is added to the IVS reaction before 4 days of incubation have passed.

In some embodiments, the added cytokine is a cytokine driving expansion of CD4⁺ T cells, such as IL2, IFNγ, IL4, IL6, IL21, IL23, and TGFB.

In some embodiments, the added cytokine is IL2.

In some embodiments, IL2 is the only cytokine added during incubation of the IVS reaction. In some embodiments, IL2 is the only cytokine added in step (c).

In some embodiments, the IL2 is added during the incubation to a final concentration of at least 100 IU/ml, or 200 IU/ml. In some embodiments, the IL2 is added during the incubation to a final concentration of about 100 IU/ml-500 IU/ml, or about 200 IU/ml-400 IU/ml. In some embodiments, the IL2 is added during the incubation to a final concentration of about 300 IU/ml.

In some embodiments, the method comprises adding IL2 to a final concentration of about 300 IU/ml after at least 2 days of incubating the IVS reaction. In some embodiments, the method comprises adding IL2 to a final concentration of about 300 IU/ml after at least 3 days of incubating the IVS reaction.

In some embodiments, the IL2 concentration is maintained during the incubation at a stable concentration in the incubated IVS reaction. In some embodiments, the IL2 concentration is maintained during the incubation at a concentration of at least 100 IU/ml, at least 200 IU/ml, about 100 IU/ml-500 IU/ml, about 200 IU/ml-400 IU/ml, or about 300 IU/ml, in the incubated IVS reaction.

Maintaining the IL concentration may be done by replacing the medium every several days, or by supplementing with additional IL2 as needed. Accordingly, IL2 may first be added after 2, 3, or 4 days of incubation, but further doses of IL2 may be added during incubation, for example once every 2, 3, or 4 days, or upon splitting of the cells.

It is noted that the phrase “IL2 concentration” is intended to refer to the final concentration of the exogenous (added) IL2 in the IVS reaction, and not to IL2 possibly secreted by the cells in the IVS reaction.

In some embodiments, combinations of co-stimulatory ligands may also be added, including, for example, anti-CD28/anti-4-1BB. The ratios of these co-stimulatory ligands can be varied to effect expansion.

In some embodiments, the method further comprises isolating or enriching for reactive VSTs e.g., by flow cytometry, using the activation markers 4-1BB and/or OX40 and optionally CD3 as markers.

In some embodiments, the method further comprises isolating or enriching for reactive virus-specific CD4⁺ T cells, e.g., by flow cytometry, using CD4 and the activation markers 4-1BB and/or OX40 as markers. In some embodiments, the method further comprises isolating or enriching for reactive virus-specific CD8⁺ T cells, e.g., by flow cytometry, using CD8 and the activation markers 4-1BB and/or OX40 as markers.

In some embodiments, the one or more viruses are selected from: adenovirus (ADV), cytomegalovirus (CMV), BK virus (BKV), John Cunningham virus (JC), Epstein-Barr virus (EBV), human herpes virus 6 (HHV6), human immunodeficiency virus (HIV), and any combination thereof.

In some embodiments, the present invention provides a method for obtaining isolated T cells specific for one or more viral antigens derived from one or more viruses, the method comprising the steps of:
a) obtaining precursor cells from a biological sample of a donor;
b) preparing an in vitro stimulation (IVS) reaction by adding to the precursor cells (i) stimulating antigens derived from the one or more viral antigens, or (ii) antigen presenting cells (APCs) presenting peptides derived from the one or more viral antigens;
c) incubating the IVS reaction in a volume of about 0.5-10 ml for about 2 hours to obtain virus-specific T cells (VSTs); and
d) incubating the VSTs in a volume of about 100-500 ml for about 8-12 days.

In some embodiments, the present invention provides a method for obtaining isolated T cells specific for one or more viral antigens derived from one or more viruses, the method comprising the steps of:
a) obtaining precursor cells from a biological sample of a donor;
b) preparing an in vitro stimulation (IVS) reaction by adding to the precursor cells (i) stimulating antigens derived from the one or more viral antigens, or (ii) antigen presenting cells (APCs) presenting peptides derived from the one or more viral antigens; and
c) incubating the IVS reaction to obtain virus-specific T cells (VSTs), wherein IL2 is added after about 2-4 days of incubation to a final concentration of about 200-400 IU/ml. In some embodiments, the present invention provides a method for obtaining isolated T cells specific for one or more viral antigens derived from one or more viruses, the method comprising the steps of:
a) obtaining precursor cells from a biological sample of a donor;
b) preparing an in vitro stimulation (IVS) reaction by adding to the precursor cells (i) stimulating antigens derived from the one or more viral antigens, or (ii) antigen presenting cells (APCs) presenting peptides derived from the one or more viral antigens;
c) incubating the IVS reaction in volume of about 0.5-10 ml for about 2 hours to obtain virus-specific T cells (VSTs); and
d) incubating the VSTs in a volume of about 100-500 ml for about 8-12 days, wherein IL2 is added after about 2-4 days of incubation to a final concentration of about 200-400 IU/ml.

In some embodiments, the present invention provides isolated T cells specific for one or more viral antigens prepared by the methods described herein.

In some embodiments, the present invention provides isolated T cells specific for one or more viral antigens prepared by a method comprising the steps of:
a) obtaining precursor cells from a biological sample of a donor;
b) preparing an IVS reaction by adding to the precursor cells (i) stimulating antigens derived from the one or more viral antigens, or (ii) antigen presenting cells (APCs) presenting peptides derived from the one or more viral antigens; and
c) incubating the IVS reaction of step (b) to obtain virus-specific T cells.

These isolated T cells (or VSTs) may be either stored, e.g., by cryopreservation, or used for treating a subject in need, such as a subject infected by a virus, as described herein.

In some embodiments, the cells are cryopreserved.

In some embodiments, the cryopreserved cells comprise at least 20%, 30%, 40%, 50%, or 60% reactive cells. In some embodiments, the percentage of reactive cells is the percentage of cells expressing 4-1BB, and/or OX40.

It is noted that the VSTs prepared by the methods described herein are the same VSTs described hereinabove, and all embodiments disclosed with reference to the VSTs of the invention (or to the isolated T cells specific for one or more viral antigens of the invention) also apply to the VSTs prepared by the methods described herein.

In some embodiments, the present invention provides methods of treating a disease caused by a virus, the methods comprising administering to a subject infected with the virus a therapeutically effective amount of the composition described herein or the isolated T cells described herein.

In some embodiments, the present invention provides methods of preventing a disease caused by a virus, the methods comprising administering to a subject at risk for being infected with the virus a therapeutically effective amount of the composition described herein or the isolated T cells described herein.

In some embodiments, the present invention provides methods of treating a disease caused by a virus, the methods comprising administering to a subject infected with the virus a therapeutically effective amount of a composition comprising isolated T cells specific for one or more viral antigens derived from the virus.

In some embodiments, the present invention provides methods of treating a disease caused by a virus, the methods comprising administering to a subject infected with the virus a therapeutically effective amount of isolated T cells specific for one or more viral antigens derived from the virus.

In some embodiments, the present invention provides methods of treating a disease caused by a virus, the method comprising administering to a subject infected with the virus a therapeutically effective amount of isolated T cells prepared by the methods described herein.

In some embodiments, the present invention provides methods of treating a disease caused by a virus, the method comprising administering to a subject infected with the virus a therapeutically effective amount of isolated T cells specific for one or more viral antigens derived from the virus, prepared by a methods comprising:
a) obtaining precursor cells from a biological sample of a donor;
b) preparing an IVS reaction by adding to the precursor cells (i) stimulating antigens derived from the one or more viral antigens, or (ii) antigen presenting cells (APCs) presenting peptides derived from the one or more viral antigens; and
c) incubating the IVS reaction of step (b) to obtain virus-specific T cells.

It is noted that since the terms “composition described herein”, “isolated T cells described herein”, and “methods described herein”, reference the compositions, isolated T cells, and methods of the invention as described herein above in more detail, all embodiments described above with respect to these terms also apply to the respective term mentioned here with respect to the methods of treatment.

In some embodiments, the virus is selected from: adenovirus (ADV), cytomegalovirus (CMV), BK virus (BKV), John Cunningham virus (JC), Epstein-Barr virus (EBV), human herpes virus 6 (HHV6), and human immunodeficiency virus (HIV).

In some embodiments, the administration is following transplantation of an organ or cells from a transplantation donor, such as a hematopoietic stem cell transplantation (HSCT) or a solid organ transplantation (SOT).

In some embodiments, the administration is part of an adoptive cell therapy (ACT).

In some embodiments, the composition is for use in ACT against virally infected cells.

In some embodiments, the T cells are autologous or allogeneic to the subject.

In some embodiments, the T cells are autologous to the subject. In some embodiments, the T cells are allogeneic to the subject. In some embodiments, the T cells are HLA-matched to or haploidentical with the subject.

According to some embodiments, there is provided a method of treating a disease caused by a virus selected from ADV, CMV, BKV, JC, EBV, HHV6, and/or HIV, the method comprising administering to a subject infected with the virus a therapeutically effective amount of a composition comprising autologous T-cells specific for one or more viral antigens derived from the virus, wherein the autologous T-cells are obtained by a method comprising the steps of:
a) obtaining precursor cells from a biological sample of the subject;
b) preparing an IVS reaction by adding to the precursor cells (i) stimulating antigens derived from the one or more viral antigens, or (ii) antigen presenting cells (APCs) presenting peptides derived from the one or more viral antigens; and
c) incubating the IVS reaction of step (b) to obtain virus-specific T cells.

The administration of the T cells and/or compositions comprising them may be conducted by any suitable method, such as, but not limited to intravenous or subcutaneous infusion, such as bolus infusion, guiding infusion, periocular infusion, subretinal infusion, intravitreal infusion, transmural infusion, coarctation infusion, Intravenous infusion, sub-conjunctival injection, subconjunctival injection, intrathoracic injection, posterior infusion, periocular infusion, or hindlimb transmission.

In some embodiments, the compositions may be administered by parenteral, intrapulmonary, or intranasal administration. Parenteral infusions include intramuscular, intravenous, intraarterial, intraperitoneal, intrathecal, intracranial, or subcutaneous administration.

In some embodiments, the administration is an intravenous administration. In some embodiments, the administration is by an intravenous injection or infusion.

In some embodiments, the administration is a single administration. In some embodiments, the administration comprises multiple administrations. In some embodiments, the administration comprises administration for a day or more than one day, e.g., up to 1 day, 2 days, or 3 days. In some embodiments, the administration may be repeated as needed, e.g., when viral load of the patient increases.

In some embodiments, the compositions of the invention are administered in combination with an additional therapeutic agent. In some embodiments, the therapeutic agent is an antirejection medicine or an antibiotic (such as an antiviral, antibacterial, or antifungal) agent. In some embodiments, the therapeutic agent is selected from a steroid, such as prednisone or an equivalent, a kinase inhibitor, e.g., a Janus kinase inhibitor such as ruxolitinib, an antirejection medicine such as mycophenolate mofetil, and an antiviral agent such as ganciclovir.

The term “treating”, as used herein, refers to means of obtaining a desired physiological effect, in this case, partially or completely curing the infection and/or symptoms thereof. The term may relate to ameliorating or inhibiting the infection, i.e. arresting its development or curing it completely by eradicating the virus.

The term “preventing”, as used herein, refers to causing the viral infection or symptoms thereof not to appear in the subject, or delaying the onset of the viral infection or symptoms thereof, such that they do not appear at the time they are expected to appear in similar cases, or causing the viral infection or symptoms thereof to appear at a diminished level.

The term “therapeutically effective amount” as used herein means an amount of the composition or of the VSTs that will result in a suitable amelioration or eradication of the viral injection. The amount must be effective to achieve the desired therapeutic effect as described above, depending inter alia on the type and severity of the infection, and the treatment regime. The therapeutically effective amount is typically determined in appropriately designed clinical trials (dose range studies) and the person skilled in the art will know how to properly conduct such trials to determine the effective amount. As generally known, an effective amount depends on a variety of factors including the affinity of the ligand to the receptor, its distribution profile within the body, a variety of pharmacological parameters such as half-life in the body, on undesired side effects, if any, and on factors such as age and gender, etc.

According to some embodiments, there is further provided a library comprising a plurality of compositions, each as described herein above, wherein the library comprises at least two compositions for which: the isolated T cells in both compositions are specific for viral antigens derived from the same virus, but the viral antigens in one composition are restricted by a different HLA type than in the other composition.

According to some embodiments, the library further comprises at least two compositions in which: the isolated T cells in one composition are specific for viral antigens derived from a virus different than the virus from which viral antigens are derived in the second composition, but the viral antigens are restricted by a same HLA type in both compositions.

It is noted that the compositions described herein as part of the library are described above in more detail. Accordingly, all embodiments described above with respect to the compositions of the invention also apply to the compositions which form part of the library.

The library is intended to be an “off-the shelf” library, or a biobank, which comprises VSTs prepared against viral antigens from different sources, and with precursor cells from different individuals in order to provide various HLA-type restrictions. Accordingly, the library comprises VSTs against antigens derived from various different viruses, such as ADV, CMV, BKV, JC, HHV6, and/or HIV. Further, the library also comprises VSTs against antigens from the same viruses, but in a different HLA background, since the VSTs are from a variety of donors. This way, the library is able to provide allogeneic T cells for treating a viral infection in a subject, by providing VSTs specific to the virus, and also matching the HLA type of the subject.

As explained above, this is achieved by using precursor cells from a variety of individuals (donors) having a variety of HLA types, and challenging each precursor cell sample with viral antigens from a variety of different viruses, so as to obtain a variety of VSTs with specificities covering a range of viruses, on different HLA backgrounds.

The compositions comprising the library may be prepared by methods described herein above.

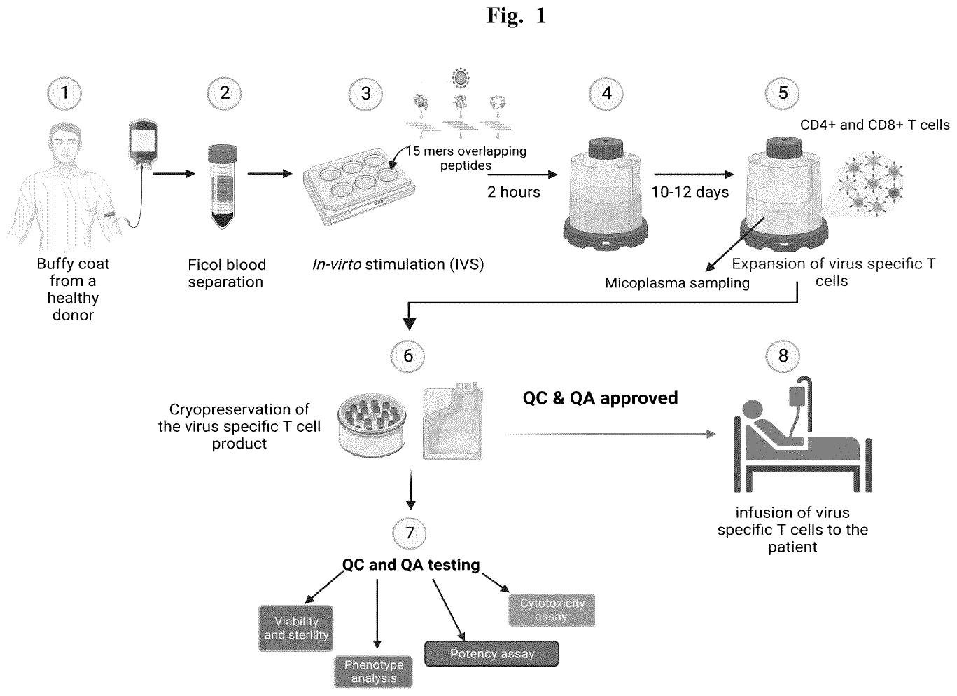

FIG. 1 depicts an outline of a method for preparing compositions including isolated T cells specific against viral antigens, according to some embodiments of the methods described herein.

As shown in FIG. 1, in some embodiments, the method of the invention is conducted by using a buffy coat containing peripheral blood mononuclear cells (PBMCs) obtained from a donor biological sample. In some embodiments, the method of the invention is conducted with donor PBMCs, or another suitable cell type as noted above, obtained by any suitable method and from any suitable blood product.

HLA typing of the donor sample is performed by any suitable method, as described herein above (e.g., by serology or by DNA typing). In some embodiments, HLA typing may take place at any time during processing of the sample or preparation of the VSTs.

In some embodiments, the donor is not infected with the virus. In some embodiments, the donor is not infected with a virus selected from ADV, CMV, BKV, JC, HHV6, and/or HIV.

The methods of the invention may use any of a variety of white blood cells, or immune cells, as precursor cells for preparing the virus-specific T cells of the invention, as explained above. In some embodiments, the precursor cells are selected from PBMCs, mononuclear cells (MNCs), or T cells. In some embodiments, the precursor cells are PBMCs.

According to the embodiments in FIG. 1, the buffy coat cells are separated on Ficoll®, and the PBMCs are plated for in vitro stimulation (IVS).

At this point, according to the embodiment of FIG. 1, viral peptides are added to the plated cells, and the mixture is incubated for 2 hours (usually at 37° C.) for priming of production of virus-specific T cells (VST) s specific for the viral peptides.

As shown in the embodiment of FIG. 1, the mixture is then transferred to a larger container, and incubated for about 10-12 days for expansion of the VSTs. In some embodiments, IL2 is added on day 3 of the incubation to a concentration of 300 IU/ml. In some embodiments, IL2 is added every 3 days.

After completion of the procedure, the VSTs are passed through a battery of tests, including tests for visibility, sterility, analysis of phenotype, reactivity, and cytotoxicity assays, for characterizing the cells.

The VSTs may be used or may be cryopreserved.

When the need arises, the VSTs may be administered to a subject in need thereof, e.g., by infusion.

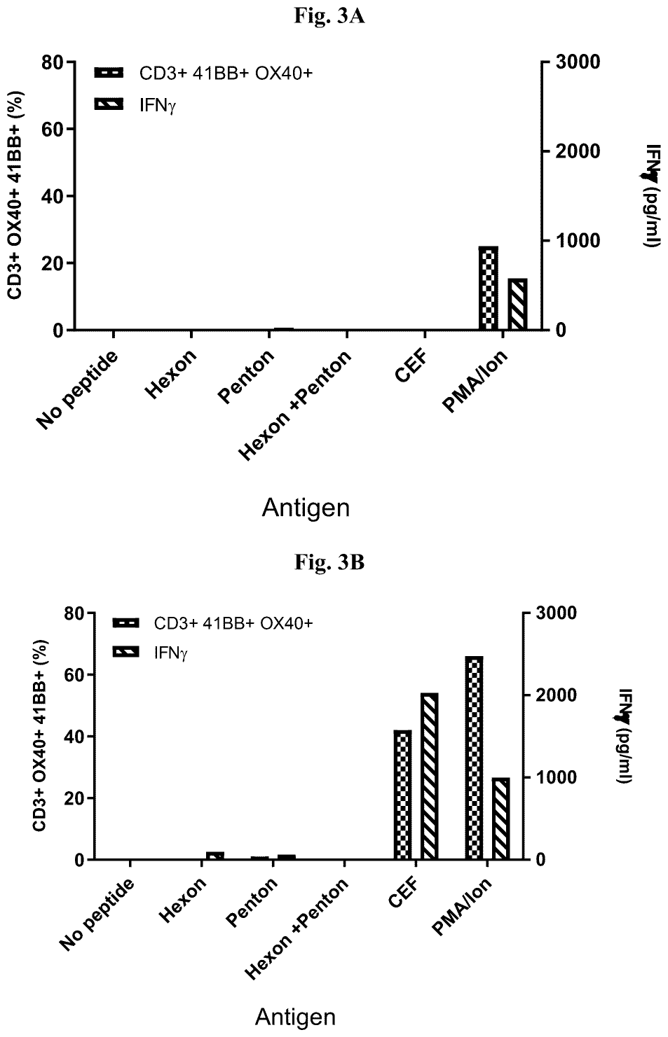

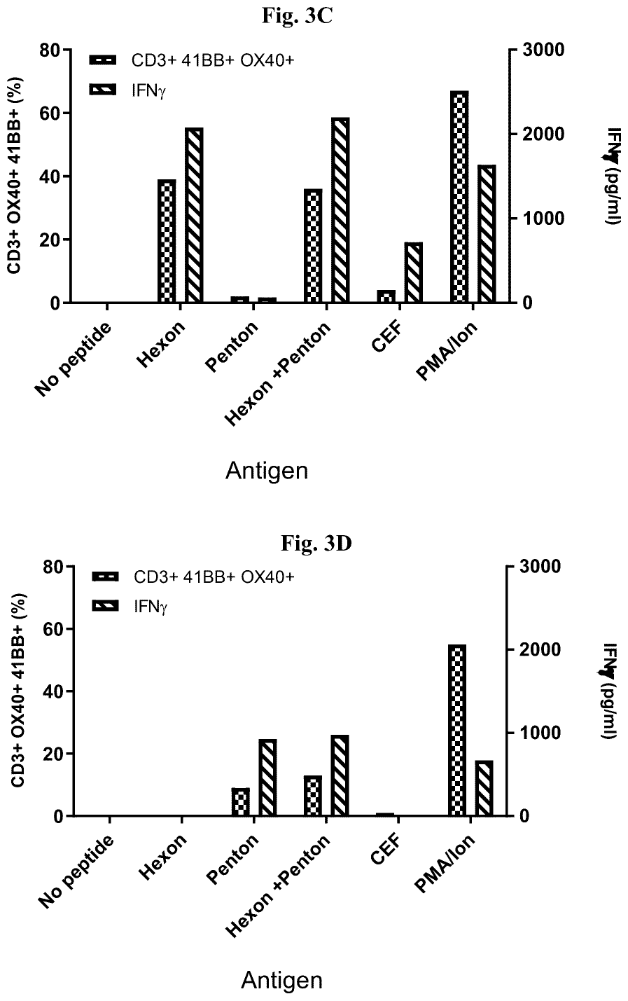

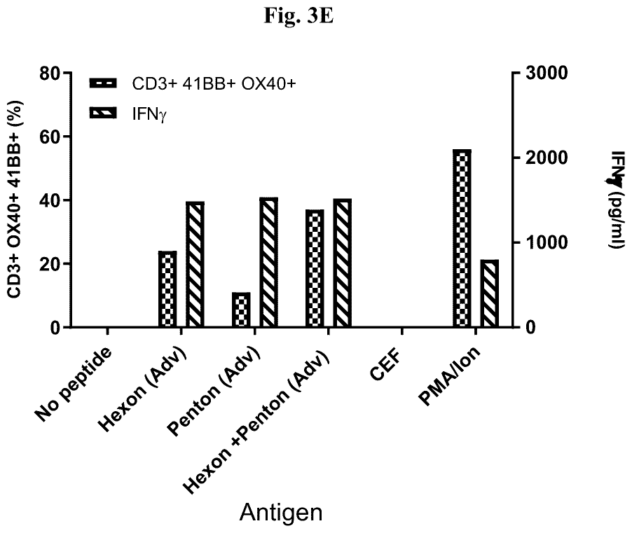

FIGS. 3A-3E demonstrate the successful preparation of VSTs, which are specific for the peptides used to generate these VSTs. As shown, FIG. 3C shows that VSTs prepared with adenovirus peptides from a hexon-library were activated in response to the hexon library and not to adenovirus peptides derived from a penton library. FIGS. 3D and 3E show a similar result for penton-specific adenoviral peptides, and from peptides from both a hexon and a penton library.

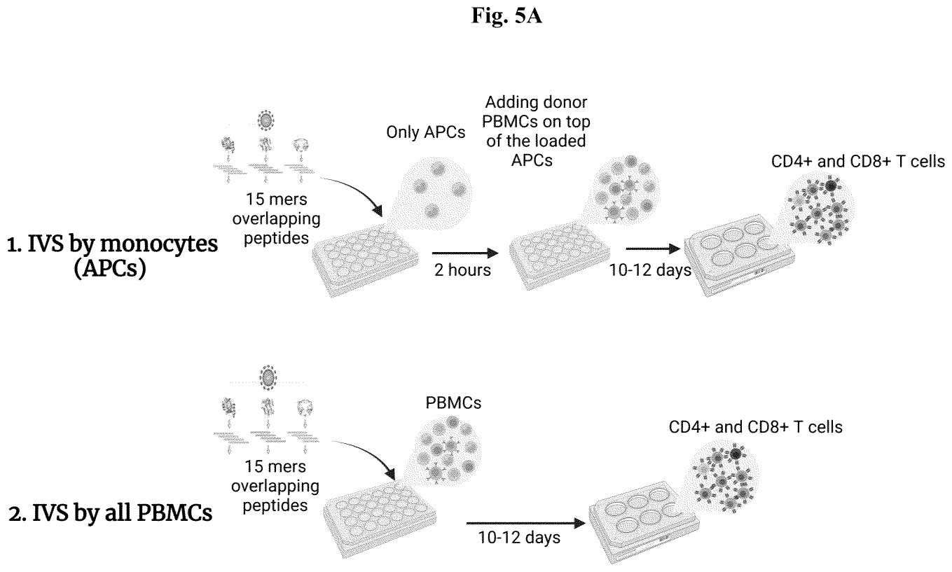

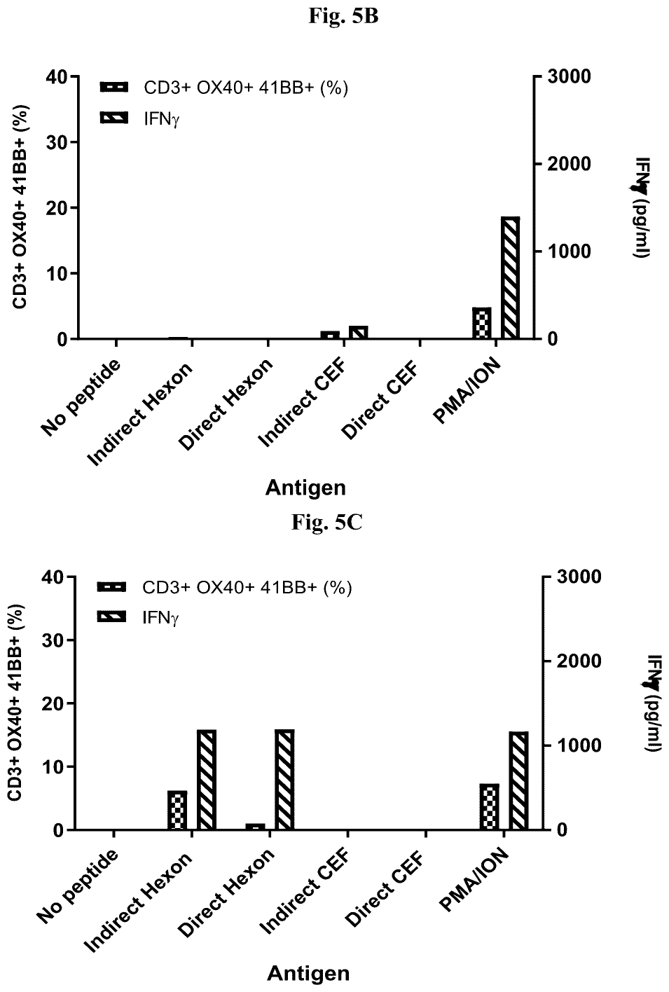

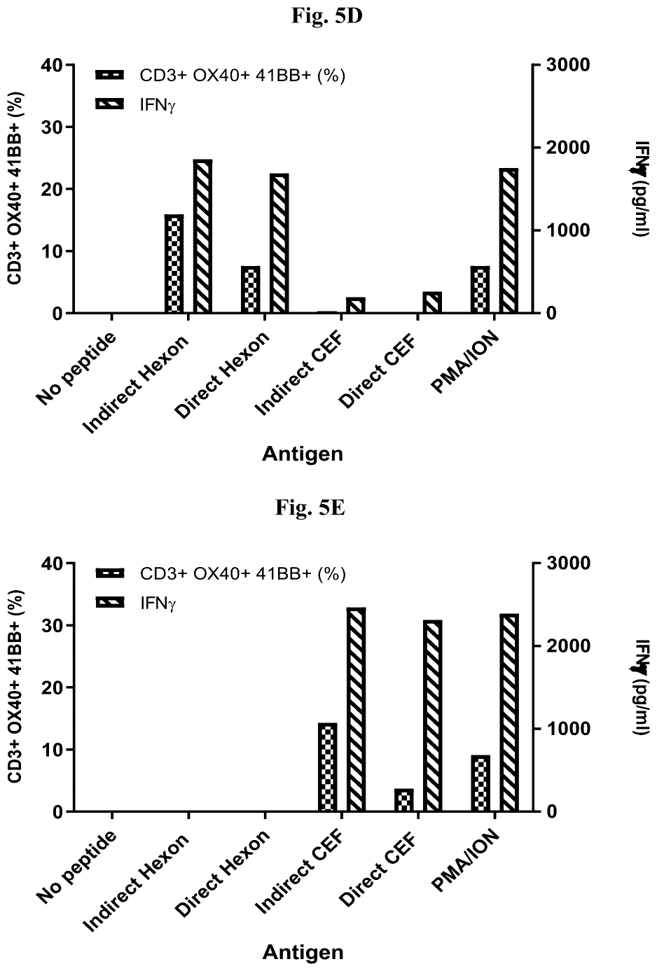

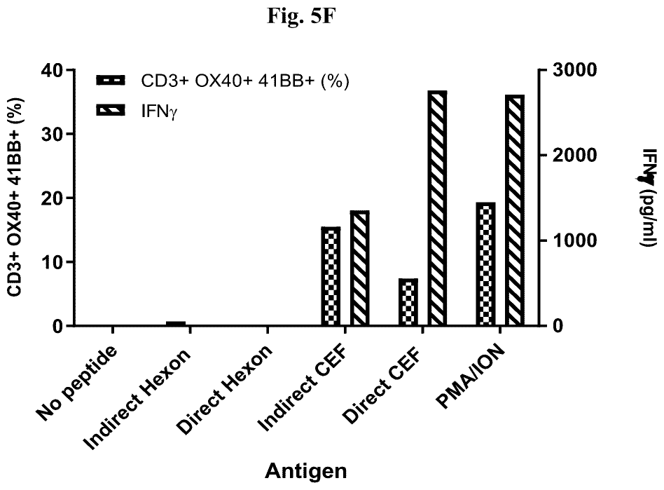

FIG. 5A shows two variations of the IVS methods, according to some embodiments. The upper panel demonstrates an “indirect IVS”. According to this embodiment, antigen presenting cells (such as dendritic cells) from a donor sample, or from an HLA-matching subject are first mixed with viral peptides and the mix is incubated, e.g., for about 2 hours. Donor PBMCs are then added to the mixture, and incubated for about 10-12 days to obtain VSTs specific for the viral peptides.

The bottom panel shows “direct IVS”. According to the shown embodiment, viral peptides are added directly to donor PBMCs, and the mixture is incubated for about 10-12 days to obtain VSTs specific for the viral peptides.

The main difference between the methods is that in the direct IVS the APCs and the T cells are together in the solution, whereas in the indirect IVS the APCs are separated and activated with the peptides before adding to the PBMCs (or T cells). Since the first (direct IVS) is simpler and faster, it was used for most purposes.

In some embodiments, not shown in this figure, in either the direct or the indirect IVS, the PBMCs may be primed by incubation of about 2 hours with the viral peptides (or APCs presenting them) in a small volume of several ml (such as about 2 ml) prior to transfer to the 10-12 days incubation in a large volume of about 300 ml.

FIGS. 5B-5F, again show the specificity of the VSTs prepared by either the direct or the indirect IVS to peptides used to prepare them. As shown, FIGS. 5C-D show that VSTs prepared with adenovirus peptides from a hexon-library, by an indirect (FIGS. 5C) or a direct (FIGS. 5D) IVS, were activated in response to the adenoviral hexon library and not to peptides derived from a CEF peptide pool, while FIGS. 5E (indirect) and 5F (direct) show a similarly specific response to a CEF peptide pool.

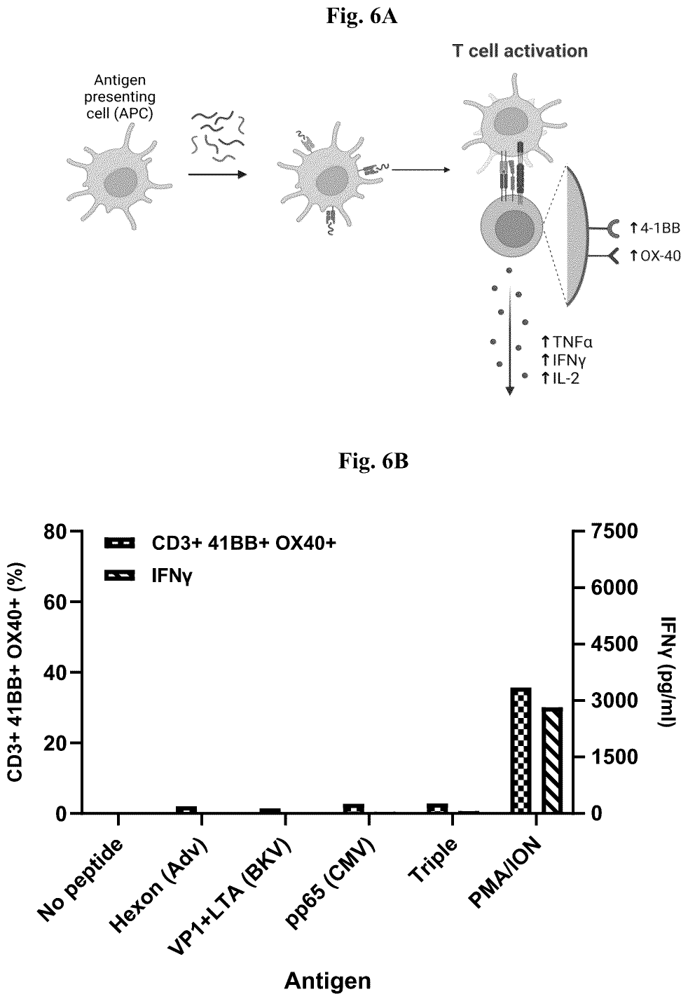

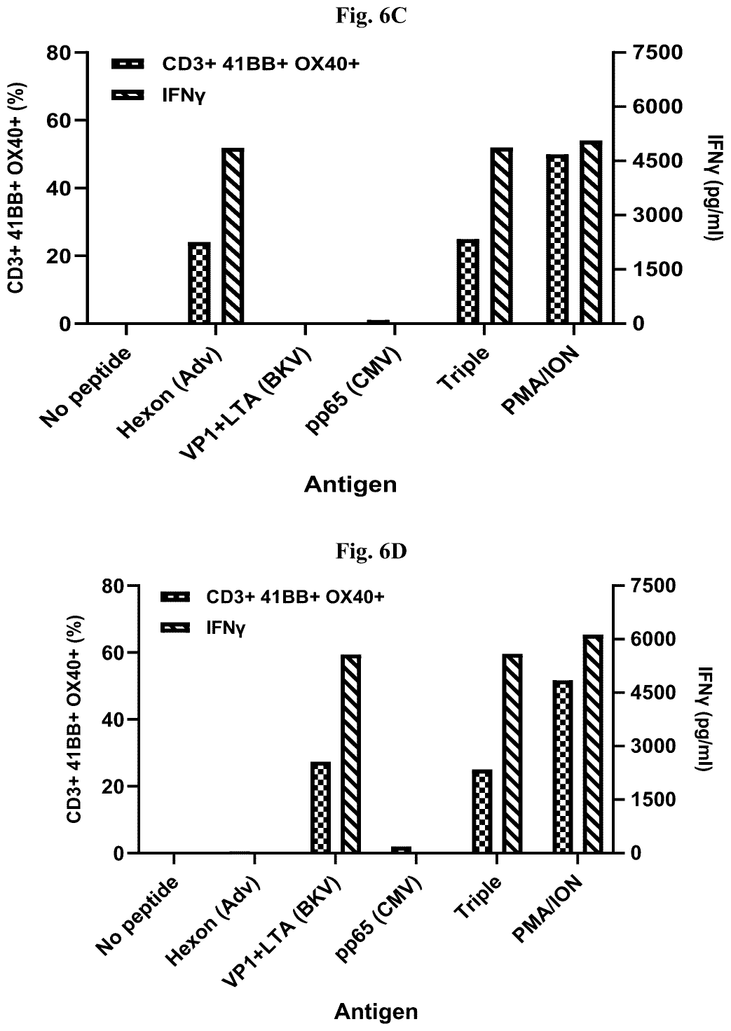

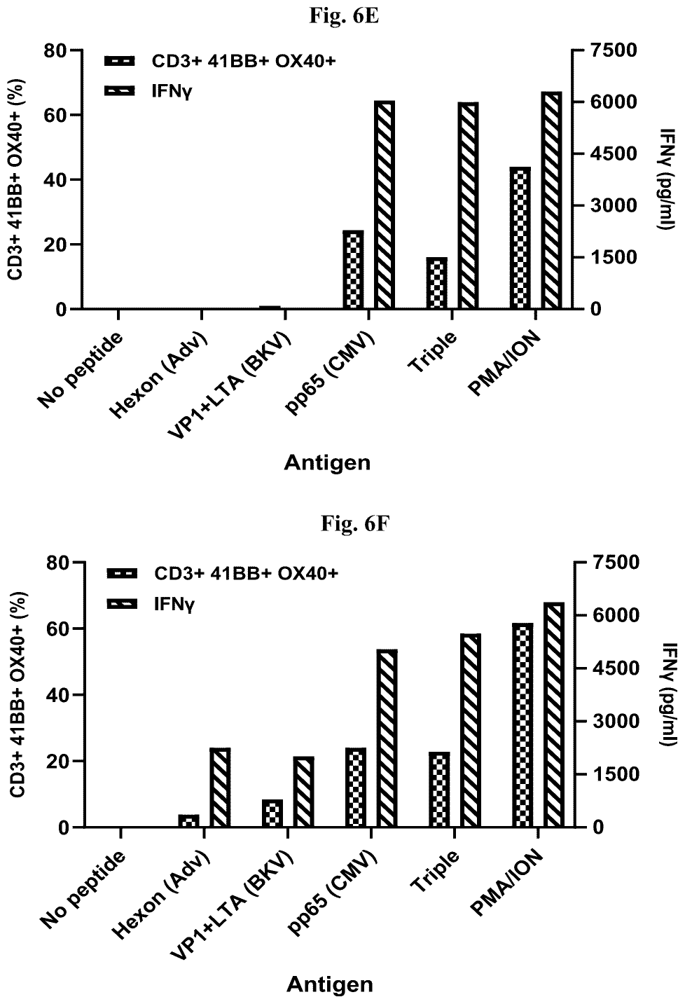

FIGS. 6B-6F show results of a triple activation of VSTs with three libraries (ADV hexon library, BKV VP1+LTA library, and CMV pp65 library). As shown, each of the individual figures shows the specificity of VSTs obtained to the peptides used to prepare them. FIGS. 6C, 6D, and 6E show specific responses of VSTs prepared with a single library to peptides of the same library (ADV, BKV, and CMV peptides, respectively), and FIG. 6F shows a response of PBMCs who were prepared with all three libraries and indeed were also reactive to all three viral peptide libraries.

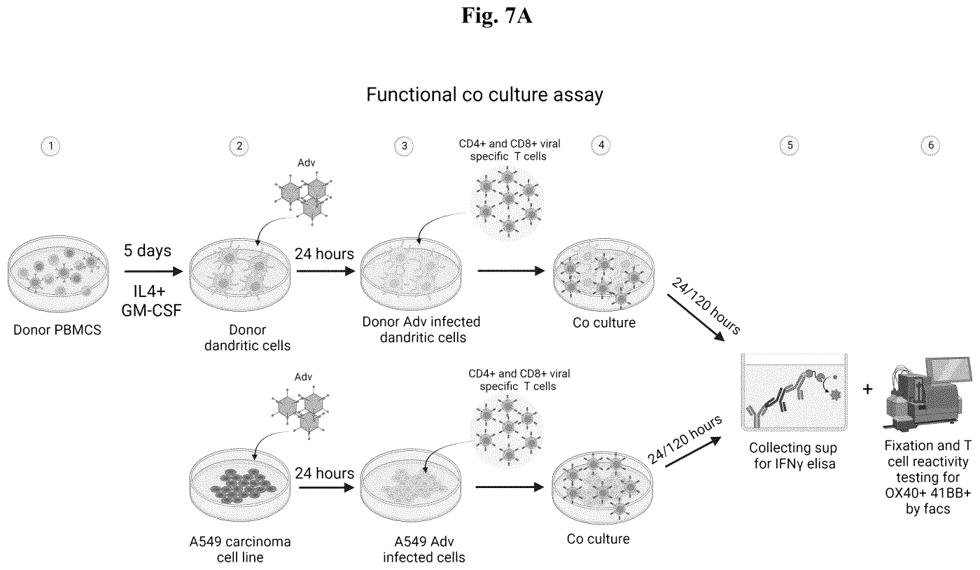

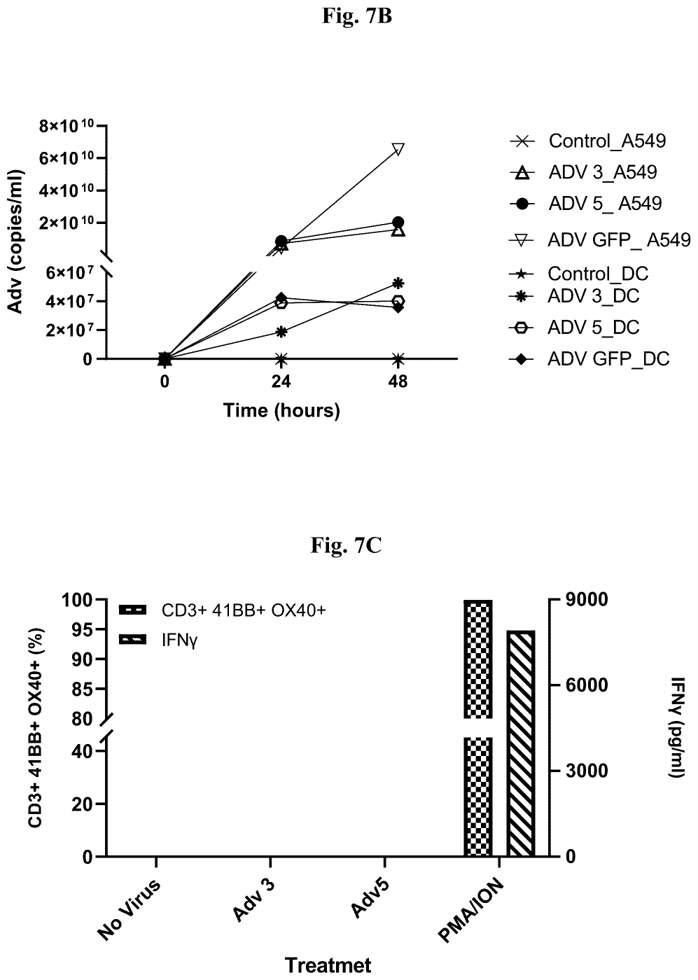

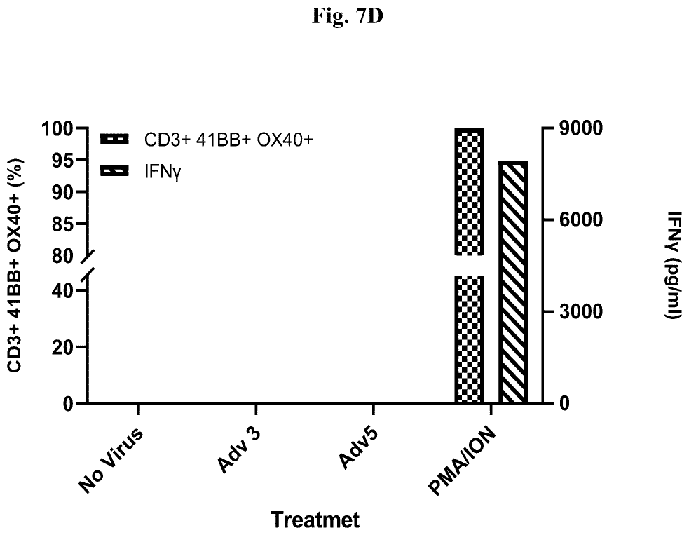

FIG. 7 shows an experiment demonstrating the importance of an HLA matching between the donor and the acceptor cells, for generating an anti-viral response. FIG. 7A, shows donor derived dendritic cells (APCs, upper panel) and A549 carcinoma cell line (bottom panel) that were both infected with a virus and then co-cultured with donor VSTs. The T cells were then tested for reactivity. As seen in FIG. 7D, in the sample where donor APCs were used, the donor (autologous) VSTs were activated. However, as seen in FIG. 7C, in the sample of the A549 cell line, the lack of HLA match caused the VSTs not to be activated.

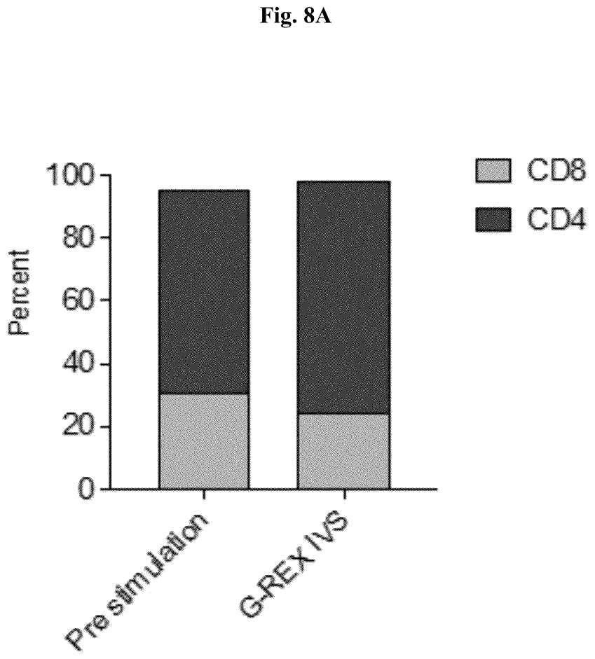

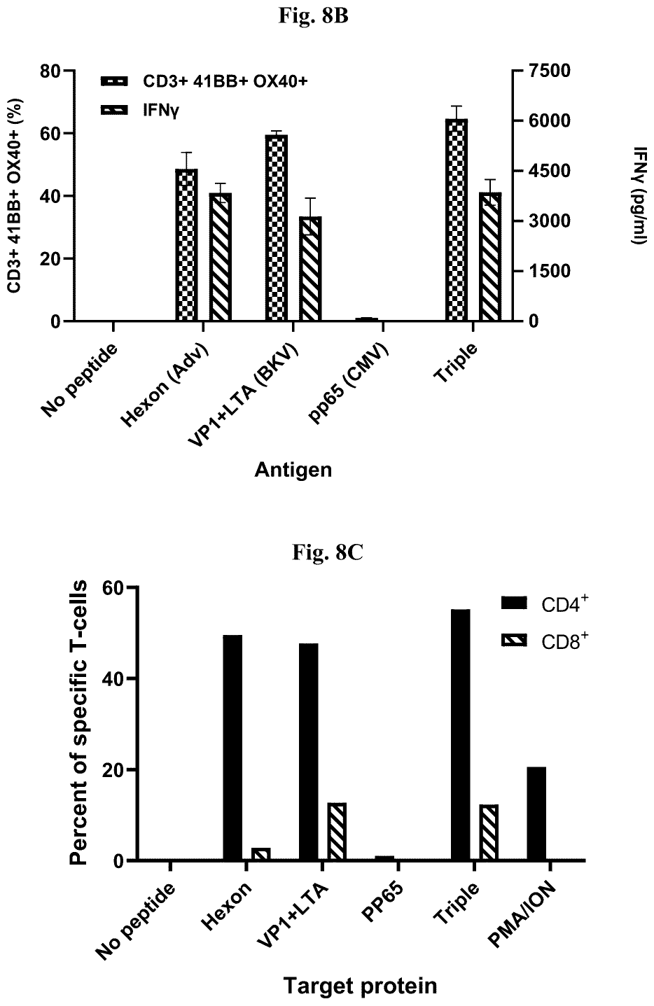

FIG. 8 shows the reactivity of the cells following IVS, and specifically the contribution of CD4⁺ cells to the reactivity. As shown in FIG. 8A, after stimulation about 97% of the total cells were T cells, about 25% were CD8+T cells and about 70% were CD4⁺ T cells. As shown in FIG. 8B, the % of reactive cells out of the total cells was higher than 40%, as measured by the % of cells expressing the activation markers 4-1BB and OX40. FIG. 8C shows the % of reactive cells out of total CD4⁺ T cells, and % of reactive cells out of total CD8+T cells. As can be seen, more than 50% of the CD4⁺ T cells were reactive, while less than 20% or even 10% of the CD8⁺ cells were reactive. Considering the relative percentage of CD4⁺ and CD8⁺ T cells in the composition (about 70% and about 30% respectively), it follows that about 35% of the cells in the composition are reactive CD4⁺ T cells, compared to less than 5% reactive CD8⁺ T cells. Accordingly, there are about 7-fold more reactive CD4⁺ T cells than reactive CD8⁺ T cells in the composition.

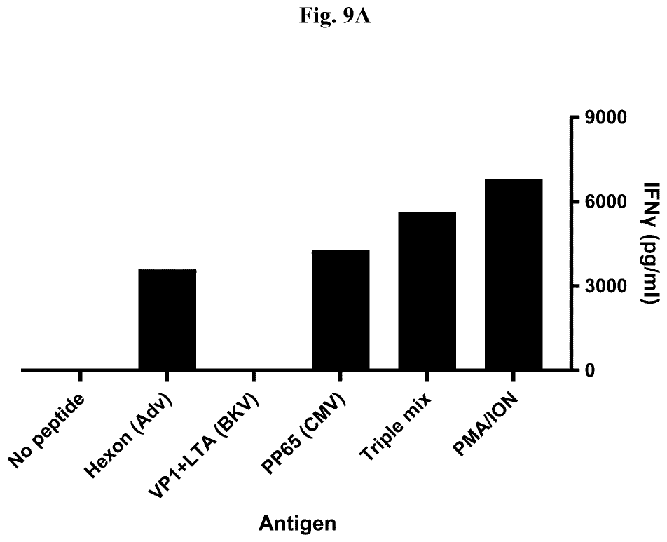

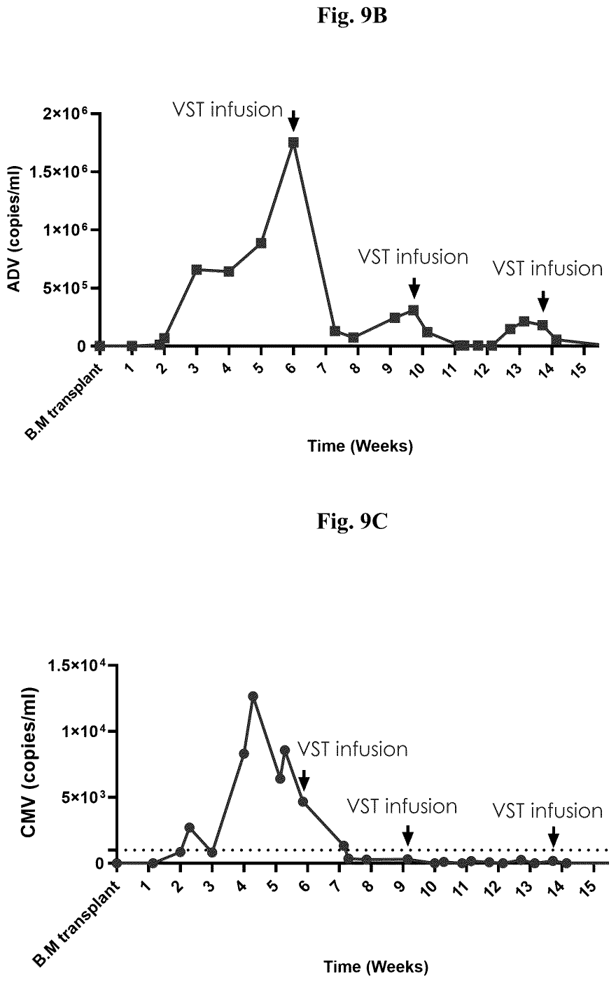

FIGS. 9A and 9B show viral load in a patient infected with a virus (ADV in FIG. 9A, CMV in FIG. 9B) following BMT, who was treated with VSTs prepared against the virus from the BMT transplantation donor. As can be seen, the treatment directly reduced the viral load each time it was administered to the patient.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### In Vitro Stimulation and Enrichment for Pathogen-Specific T Cells

In order to stimulate and enrich for pathogen-specific T cells from healthy donors, PBMCs were isolated and monocyte-derived dendritic cells were produced to serve as antigen-presenting cells (APCs).

The produced APCs were loaded with a commercial CEF T cell stimulation pool (CEFX™, JPT Peptide Technologies) composed of 175 known HLA class I epitopes derived from both bacteria and viruses, including cytomegalovirus (CMV), Epstein-Barr virus (EBV), and influenza virus. PBMCs were in vitro stimulated (IVS) with CEF-loaded APCs for seven days in the presence of IL2 (IVS 1). The cells were then restimulated for the second time (IVS 2) for seven more days.

To test for the level of CEF-specific T cells, each IVS sample was co-cultured with APCs loaded with CEF and the expression of the T cell activation marker 4-1BB on the surface of CD8 T cells was measured. APCs without CEF and non-activated PBMC were served as controls.

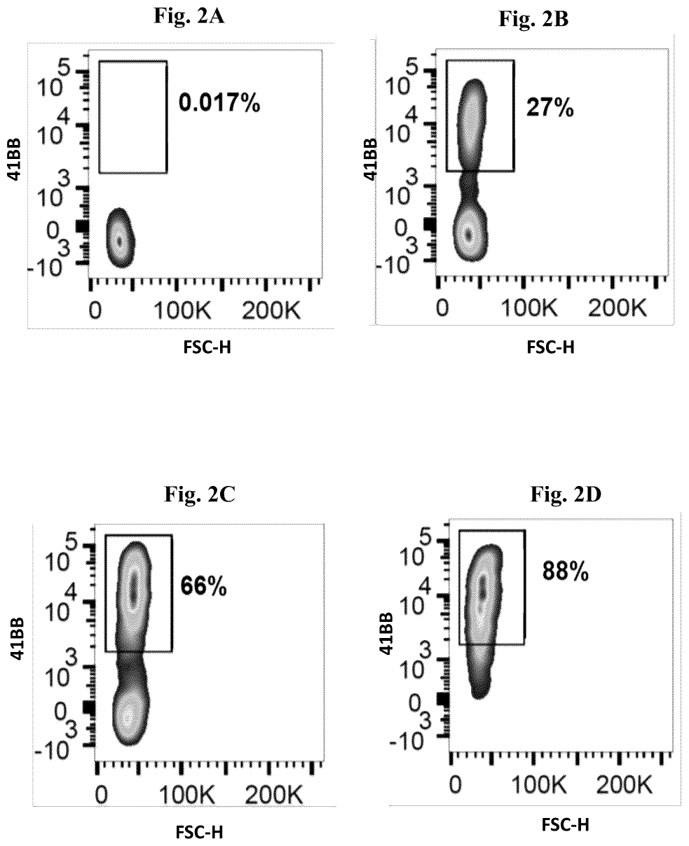

As can be seen in the results presented in FIG. 2, in the initial PBMC culture, 27% of the cells were CEF specific (FIG. 2B), and following IVS1, the level of specific cells increased to 66% (FIG. C). Additional enrichment was observed following the second stimulation IVS2 (88%) (FIG. 2D). The results presented in FIG. 2 clearly show the ability to stimulate and enrich pathogen-specific T cells.

### Example 2

### In Vitro Stimulation With Peptides of ADV or CMV

TABLE 1
Reagents

| Reagent | Catalog | manufacturer/Distributer |
|---|---|---|
| Ficoll-Paque ® | 95021-207 | GE healthcare/Daniel |
| PREMIUM | | biotech |
| RPMI1640 | 01-104-1A | Biological industries |
| AIM-V ™ medium | 12055091 | Gibco/Rhenium |
| (Research grade) | | |
| Human serum | H-4522-10ML | Sigma |
| L-Glutamine | 03-020-1B | Biological industries |
| Pen/Strep (penicillin, | 03-031-1B | Biological industries |
| 10⁴ U/ml, streptomycin, | | |
| 10⁴ μg/ml) | | |
| Recombinant human | CH1001 | Megapharm, Chiron |
| IL2 | | Novartis, or Proleukin ® |
| | | (aldesleukin) (drug) |
| Hexon (ADV) peptide | 170-076-106 | Miltenyi/Almog |
| library (clinical grade) | | |
| Penton (ADV) library | | Miltenyi/Almog |
| PP65 (CMV) peptide | 170-076-109 | Miltenyi/Almog |
| library (clinical grade) | | |

Cells were prepared from 30 ml of a blood sample from a donor.

Blood samples were diluted two-fold with MACS® buffer, and PBMCs were isolated on Ficoll®, and resuspended in PBS. PBMCs were then centrifuged and resuspended in VST medium containing 2 mM L-glutamine, 5% human serum in AIM-V™ medium, plated in 24 well plates at 2×10⁶ PBMCs per 250 μl medium, and incubated at 37° C., 5% CO₂ for 2 hours.

For preparing antigen-presenting APCs, one hour before the incubation ends, the desired peptides were prepared at 250 μl/per sample to a final concentration of 1 μg/ml, and were added to the plates in triplicates.

For obtaining T cells, 10×10⁶ PBMCs in VST medium from the same sample were separated by using magnetic beads (e.g., by Pan T Cell MicroBeads (Miltenyi Biotech)), according to the manufacturer's instructions.

For IVS, 2×10⁶ T cells were added to the APCs in each well in a final volume of 1 ml, and the cells were incubated at 37° C., 5% CO₂ for 72 hours.

Cells were then fed by adding 1 ml of VST medium with IL2 (600 IU/ml) to each well (to a final concentration of 300 IU/ml) and incubating at 37° C. for 72 h. Cells were further fed and split as needed. Cells were incubated during IVS with all peptides for 12-15 days to enrich for peptide-specific T cells.

To test for the level of ADV-specific T cells, each IVS sample was co-cultured with APCs loaded with penton, hexon or CEF and the expression of the T cell activation markers OX40 and 4-1BB on the surface of T cells were measured by fluorescence cell sorter (FACS), and the level presented in FIGS. 3A-3E demonstrate that the IVS enriched for ADV specific T cells to a level of 20-60% of the T cell culture. Furthermore, the enriched ADV-specific T cells showed specificity to both the hexon and penton peptides and not to the unloaded or CEF controls. FIG. 3C shows hexon (ADV)-specific response for the hexon-IVS PBMCs, FIG. 3D shows penton (ADV)-specific response for the penton-IVS PBMCs, and FIG. 3E shows a response against both hexon and penton peptides for a divalent (hexon+penton) IVS PBMCs. CEF-loaded PBMCs were used as a positive control, and unloaded cells served as a negative control.

### Example 3

### Infection of Primary Donor Monocytes With Live Adenovirus (ADV)

A method to infect primary donor monocytes with live viruses was developed, in order to facilitate to functionally test the ability of the VSTs to recognize and kill autologous donor tissue infected with a relevant virus. For functional validation, the method includes isolating monocytes from donor PBMCs harboring relevant HLA molecules, infecting the monocytes with a live virus, and after 24 hours co-incubating the virus-infected monocytes with VSTs directed against the same virus. Specific T-cell activation may then be measured by secretion of IFNγ following virus recognition.

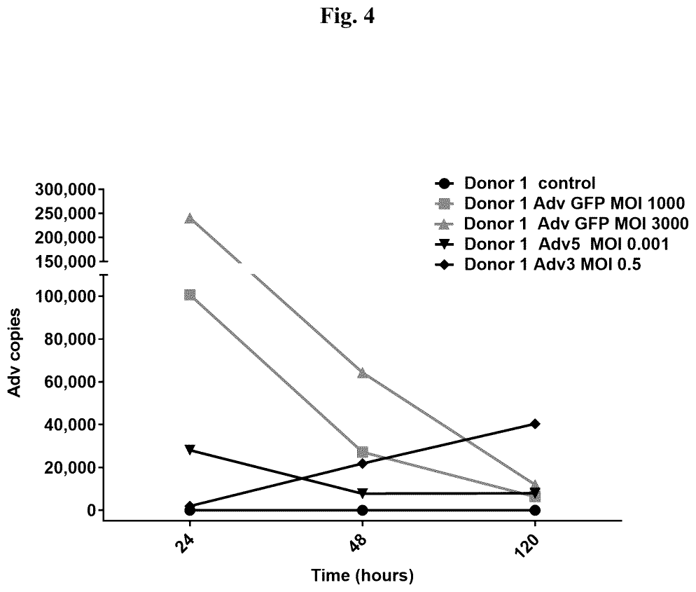

The method was tested with ADV as the virus. To establish the ADV infection procedure, three viral samples were used: an ADV-GFP system and two additional ADV subtypes (ADV5 and ADV3) isolated by the national virology laboratory from patients suffering from ADV infections. The results presented in FIG. 4 demonstrate that PBMCs can be efficiently infected with different ADV variants. The differences in ADV copies between the ADV-GFP and the ADV5/3 variants result from a lower concentration of the virus.

### Example 4

### Direct vs. Indirect In vitro Stimulation With Hexon Peptide Library

Healthy donor derived peripheral blood mononuclear cells (PBMCs) went through an indirect/direct in vitro stimulation (IVS) with a peptide library covering the adenovirus capsid hexon protein. The scheme of the IVS is shown in FIG. 5A.

Indirect IVS: As shown in FIG. 5A (top panel), donor PBMCs were plated in a tissue culture plate and allowed to attach. Unattached cells were washed, and the attached cells (antigen presenting cells-APCs) were loaded with a hexon peptide library, for presentation by APCs. Following 2 hours of incubation, the peptide library was washed and new PBMCs from the same donor were loaded on top of the APCs for in vitro stimulation. The cells were cultured for a total of 12 days, with the addition of 300 IU/ml of IL2 on day 3. Cells were split into a larger dish as necessary every 3-4 days.

Direct IVS: As shown in FIG. 5A (bottom panel), donor PBMCs were plated in a tissue culture plate. Then, a hexon peptide library was added directly to the PBMCs medium in order to cause an in vitro stimulation (IVS). Similar to the indirect IVS, the cells were cultured for a total of 12 days, with the addition of 300 IU/ml of IL2 on day 3. Cells were split into a larger dish as necessary every 3-4 days. This treatment by a 12-day culture and supplementation at day 3 with 300 IU/ml of IL2 was conducted for all IVS protocols described herein.

The product of the direct/indirect IVS was then tested for its reactivity by a co-culture with the same donor PBMCs that were loaded by different antigens (no peptide/hexon peptides/CEF HLA class I control peptide pool) in a direct/indirect manner. PMA/ION was used as positive control. The T cells were stained for CD3 and for activation markers 4-1BB and OX40 and analyzed by FACS, and the supernatant was analyzed for IFNγ level by ELISA. As shown in FIGS. 5B-5F, response is seen only when the same peptides were used for activation in the IVS and in the PBMCs. FIGS. 5C (indirect) and 5D (direct) show response to hexon peptides only by PBMCs that went through IVS with hexon peptides, while FIGS. 5E (indirect) and 5F (direct) show response to a CEF peptide pool only by PBMCs who went through IVS with a CEF peptide pool.

### Example 5

### Single and Triple IVS (against ADV, BKV, and CMV)

PBMCS went through a direct IVS (FIG. 6A shows a scheme of the IVS process) against a single or triple viral peptide library including peptides from adenovirus (ADV, capsid protein hexon peptides), BK virus (BKV, capsid protein VP1, and large T-antigen LTA peptides), and cytomegalovirus (CMV, structural protein pp65) separately or together. The cells were cultured for 12 days with IL2 supplementation at day 3 (as mentioned above in Example 4). The virus-specific T cells (VSTs) resulting from each IVS were then tested for their reactivity by a co-culture with donor APCs loaded with no peptide, hexon library (ADV), VP1+LTA (BKV), or pp65 (CMV) each separately (single), or the three viral peptide pools together (triple, i.e., hexon, VP1+LTA, and pp65). PMA/ION was used as positive control. Following 18 hours of co-culture, the T cells were stained for CD3 and for activation markers 4-1BB and OX40 and analyzed by FACS, and the supernatant was analyzed for IFNγ level by ELISA. FIGS. 6B-6F show the results of the cu-culture. The specificity of the response is clear by that only when antigens match there was a response. FIG. 6C shows response to adenovirus peptides (Hexon library) only by PBMCs who went through IVS with hexon library (alone or part of the triple IVS); FIG. 6D shows response to BKV VP1+LTA peptides only by PBMCs who went through IVS with VP1+LTA BKV peptides; and FIG. 6E shows response to CMV peptide (PP65) only by PBMCs who went through IVS with CMV peptide (PP65). FIG. 6F. shows a response of PBMCs who went through Triple IVS (peptides for ADV, BKV, and CMV) to ADV, BKV, and CMV peptides.

### Example 6

### Functional Co-Culture Validation of Virus-Specific T Cells (VSTs) Against Live ADV Strains

FIG. 7A shows a scheme of the experiment. Donor derived dendritic cells (upper panel) and A549 carcinoma cell line (bottom panel) were infected with one of two adenoviral strains ADV3 or ADV5, and co-cultured with VSTs produced as described in Example 5. the T cells were stained for CD3 and for activation markers 4-1BB and OX40 and analyzed by FACS, and the supernatant was analyzed for IFNγ level by ELISA. FIG. 7B shows the growth of the adenovirus strains in the two cell types (DC and A549). FIGS. 7C and 7D show response for the two cell types when incubated with the trivalent VSTs after 96 hours of co-culture. As can be seen from these figures, no response was obtained for the ADV-infected non-HLA-matched A549 cells with ADV3 and ADV5 (FIG. 7C), while a strong response was seen for ADV-infected dendritic cells of the donor (autologous) with ADV3 and ADV5 loaded donor PBMCs (FIG. 7D). The same results were already seen after a 24h co-culture (data not shown)

### Example 7

### Large-Scale, GMP-Compliant IVS

Large-scale IVS process against the three viruses (ADV, BKV, CMV) was conducted in a G-REX® production platform as described below.

TABLE 3
Reagents

| Reagent | Catalog | manufacturer/Distributer |
|---|---|---|
| Ficoll-Paque ® | 17544202 | Cytiva/Danyel biotech |
| PREMIUM | | |
| AIM V ™ medium | 87-0112BK | Gibco/Rhenium |
| (GMP) | | |
| Human male AB Off- | AC-002-1B-HI | Access Biologicals/ |
| Clot Serum Heat | | IMBH |
| the (Inactivated) | | |
| GlutaMAX ™ 200 mM | A1286001 | Gibco/Rhenium |
| (L-glutamine) | | |
| Recombinant human | 5060229220264 | Clinigen/Sheba |
| IL2, Proleukin® | | pharmacy |
| (aldesleukin) (drug) | | |
| Hexon (Adv) peptide | 170-076-106 | Miltenyi/Almog |
| library (clinical grade) | | |
| LTA (BKV) peptide | 170-076-139 | Miltenyi/Almog |
| library (clinical grade) | | |
| VP1 (BKV) peptide | 170-076-138 | Miltenyi/Almog |
| library (clinical grade) | | |
| PP65 (CMV) peptide | 170-076-109 | Miltenyi/Almog |
| library (clinical grade) | | |
| Human serum albumin | 901064 | BPL/Sheba pharmacy |

PBMCs from a heathy donor were isolated on Ficoll®, as described above in Example 2, and suspended in warm (37° C.) VST medium (2 mM GlutaMAX™, 5% heat inactivated human serum, in AIM-V™ medium). 150×10⁶ cells in 1,840 μl VST medium were placed in one well in a 6-well ultra-low attachment surface plate, for direct IVS.

Single peptide libraries for hexon (ADV), VP1 (BKV), LTA (BKV) and PP65 (CMV) were used to prepare a pooled peptide library master mix by mixing equal volumes from each 50 μg/ml library stock. For IVS, 160 μl of the pooled peptide library master mix were added to the well containing the PBMCs, and the cells were incubated at 37° C., with 5% CO₂ for 2 hours.

After 2 hours of priming, the activated cells were transferred to a G-REX® 100M production platform (Wilson Wolf, Cat. #P/N 81100) containing 295 ml warm VST medium (37° C.), wells were washed three times with 1 m of VST medium each, the washing volume was added to the G-REX® (final volume of 300 ml), and the cells were incubated for 3 days at 37° C., with 5% CO₂, without adding IL2. After 3 days, warm VST medium containing IL2 was added to the G-REX®, to a final concentration of 300 IU/ml, and the culture continued to grow for 7-9 days at 37° C., with 5% CO₂.

The VSTs were resuspended and collected from the G-REX® membrane. After centrifugation, the cells were resuspended in PBS containing 5.7% human serum albumin. Cells were then counted and analyzed by FACS. 20-50×10⁶ cells may be transferred to freezing bags in freezing medium (DMSO-dextran) for cryopreservation.

The trivalent IVS product was estimated as 4.4×10⁹ total cells, comprising about 98% T cells. This represents about an 85-fold expansion compared to the amount of cells at the IVS step (data not shown).

As shown in FIG. 8A, the level of CD4⁺ cells increased and the level of CD8⁺ decreased following stimulation. As seen, about 97% of the cells were T cells, about 25% were CD8⁺ T cells and about 70% were CD4⁺ T cells. The VST product was tested for its reactivity against viral peptides, and following 18 hours of co-culture the T cells were stained for CD3 and the activation markers 4-1BB and OX40, and analyzed by FACS, and the supernatant was analyzed for IFNγ level by ELISA. As shown in FIG. 8B, the trivalent VSTs reacted with ADV and BKV peptides but not for the CMV peptides. FIG. 8C shows the percent reactive out of total CD4⁺ T cells and percent reactive out of total CD8⁺ T cells. As can be seen, in most cases at least about 50% of the CD4⁺ T cells are reactive, while 10% or less of the CD8⁺ T cells are reactive.

When testing cryopreserved VST products, functionality, as tested by the % of cells expressing the markers CD3, 4-1BB, and OX40, appears to be lower compared to a fresh product, and to be reduced with expansion of the cells. However, cryoprecipitates at concentrations of 1-52×10⁶ cells/ml, which were produced following the process described below and tested, presented with a relatively high reactivity of up to about 80-90% compared to fresh cells, against the stimulating antigens Hexon, VP1+LTA, or the triple pool including antigens from ADV (Hexon), BKV (VP1+LTA), and CMV (PP65). While the fresh cells showed reactivity of about 50-60%, the cryoprecipitates showed reactivity of between 20% and 55%. The highest reactivity was measured for a cryoprecipitate of 2×10⁶ cells/ml.

### Example 8

### Treatment of a Patient After Bone Marrow Transplantation With the VSTs of the Invention

A pediatric patient following a haploidentical BM transplantation presented with high serum levels of ADV and CMV. VSTs specific for ADV and CMV were prepared by direct IVS, as described in Example 5, from the donor PBMCs. The reactivity of the VSTs was tested as described above, and shown in FIG. 9A. The anti-ADV and anti-CMV VSTs were administered at 5×10⁴/kg to the patient by infusion at three time points (during weeks 6, 9, and 14). As can be seen from FIG. 9B and 9C, the viral load for both ADV (FIG. 9B) and CMV (FIG. 9C) decreased after each VST infusion, and went down to about 0. It is noted that the patient was treated for CMV also by other drugs (ganciclovir). Control of the ADV levels was achieved for about 70 days.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

1.-46. (canceled)

47. A composition comprising isolated T cells specific for one or more viral antigens derived from one or more viruses, wherein at least 60% of the cells in the composition are CD4⁺ T cells and/or at most 30% of the cells in the composition are CD8⁺ T cells.

48. The composition of claim 47, comprising CD4⁺ T cells and CD8⁺ T cells at a ratio of between about 2:1 and about 4:1 CD4⁺ to CD8+.

49. The composition of claim 47, wherein at least 40% of the cells, at least 40% of the CD4⁺ T cells, and/or at most 20% of the CD8⁺ T cells in the composition are reactive T cells, specific for the one or more viral antigens.

50. The composition of claim 47, wherein the one or more viruses are selected from the group consisting of: adenovirus (ADV), cytomegalovirus (CMV), BK virus (BKV), John Cunningham virus (JC), Epstein-Barr virus (EBV), human herpes virus 6 (HHV6), human immunodeficiency virus (HIV), and any combination thereof.

51. The composition of claims 47, wherein the isolated T cells specific for one or more viral antigens do not comprise T cells expressing a chimeric antigen receptor (CAR) or a recombinant T cell receptor (TCR).

52. The composition of claim 47, further comprising a pharmaceutically acceptable carrier.

53. A method of treating a subject infected with a virus, or for preventing a viral infection in a subject at risk, the method comprising administering to the subject a therapeutically effective amount of the composition of claim 52.

54. The method of claim 53, wherein the virus is selected from the group consisting of:
adenovirus (ADV), cytomegalovirus (CMV), BK virus (BKV), Epstein-Barr virus (EBV), human herpes virus 6 (HHV6), John Cunningham virus (JC), and human immunodeficiency virus (HIV).

55. The method of claim 53, wherein the administration is following transplantation of an organ or cells from a transplantation donor, such as a hematopoietic stem cell transplantation (HSCT) or a solid organ transplantation (SOT).

56. The method of claim 53, wherein the treating or preventing is conducted by adoptive cell therapy (ACT).

57. A method for obtaining isolated T cells specific for one or more viral antigens derived from one or more viruses, the method comprising the steps of:
a) obtaining precursor cells from a biological sample of a donor;
b) preparing an in vitro stimulation (IVS) reaction by adding to the precursor cells (i) stimulating antigens derived from the one or more viral antigens, or (ii) antigen presenting cells (APCs) presenting peptides derived from the one or more viral antigens; and
c) incubating the IVS reaction to obtain virus-specific T cells, and further comprising a priming step prior to the incubation in step (c), in which the IVS reaction is incubated for a length of about 1-5 hours in a small volume, and the IVS reaction volume is increased in step (c), following the priming step.

58. The method of claim 57, wherein the incubating of the IVS reaction in step (c) of claim 57 is conducted for a length of about 8-15 days, or about 10-12 days.

59. The method of claim 57, wherein the small volume is about 0.5-10 ml, or about 2 ml.

60. The method of claim 57, wherein the IVS reaction volume is increased in step (c) of claim 57 by about 10-500 fold, or by about 100-200-fold.

61. The method of claim 57, wherein IL2 is added to the IVS reaction during the incubation in step (c) of claim 57.

62. The method of claim 61, wherein the IL2 is added after about 2-4 days of incubation.

63. The method of claim 61, wherein IL2 is the only cytokine added to the IVS reaction during the incubation.

64. The method of claim 61, wherein IL2 is added to a final concentration of about 300 IU/ml.

65. The method of claim 57, wherein no cytokine is added to the IVS reaction before between about 2-4 days of incubation have passed.

66. The method of claim 57, wherein the donor is not pre-treated with granulocyte colony stimulating factor (G-CSF) prior to donating the biological sample.
